(19) United States
(12) Patent Application Publication
(10) Pub. No.: US 2013/0230609 A1
(43) Pub. Date: Sep. 5, 2013

(54) ANTIMICROBIAL/PRESERVATIVE COMPOSITIONS COMPRISING BOTANICALS

(71) Applicants: Shanta Modak, River Edge, NJ (US); Santoshkumar Dongre, New York, NY (US); Nayana Baiju, Kerala (IN); Hari Krishnan Ramachandran, New York, NY (US); Lauserpina Caraos, Hollis, NY (US)

(72) Inventors: Shanta Modak, River Edge, NJ (US); Santoshkumar Dongre, New York, NY (US); Nayana Baiju, Kerala (IN); Hari Krishnan Ramachandran, New York, NY (US); Lauserpina Caraos, Hollis, NY (US)

(21) Appl. No.: 13/412,464

(22) Filed: Mar. 5, 2012

(51) Int. Cl. A01N 65/08; A61K 36/18; A01N 65/22; A01N 65/28; A01N 65/24; A01P 1/00; A01P 3/00; A61P 17/10; A61P 31/10; A61K 8/97; A01N 65/44

(52) U.S. Cl. 424/739; 424/725; 424/747; 424/745; 424/742; 424/750; 424/769

(57) ABSTRACT

The present subject matter relates to a preservative or antimicrobial compositions which comprise low concentrations of botanical extracts, in synergistic combinations with alkanediols in a solvent system, optionally with fruit acids. Additionally, the present subject matter relates to a preservative or antimicrobial compositions which comprise a silver compound, an essential oil or individual constituent, one or more zinc salts, and one or more alkanediol. The compositions of the application may be used in personal care products including wound care products or in veterinary use. Preferably, the compositions of the application have little or no human-detectable fragrance.

### 1. INTRODUCTION

The compositions of the present subject matter contain combinations of low concentrations of benzyl alcohol, essential oils and botanical extracts (including plant extracts and fruit extracts), in synergistic combinations. The compositions optionally contain sesquiterpenoids, alkanediols, solvents, natural organic acids (including fruit acids), plant derived alcohols, natural gelling agents, vegetable glycerin, and anti-inflammatory agents as well. In specific embodiments, certain concentrations of the solvent benzyl alcohol have been found to exhibit synergistic antimicrobial efficacy with certain botanical acids, especially fruit acids.

The present subject matter additionally relates to compositions containing silver sulfadiazine, an antimicrobial agent and calendula oil, a wound healing agent. The compositions optionally contain silver releasing agents and/or antifungal activity enhancers. The compositions may be used in topical hydrophilic creams for the treatment of burns, wounds, and surface infections.

Preferably, the inventive compositions have very mild or little to no fragrance. The compositions of the application may be used as non-toxic, non-fragrant alternatives to conventional preservatives or may be combined with other antimicrobial agents to enhance their activity, and may be particularly useful in personal care and veterinary product applications.

### 2. BACKGROUND

Essential oils are volatile oils obtained from plant or animal sources and are composed of complex mixtures of several constituents, such as monoterpenes and sesquiterpene hydrocarbons, monoterpene and sesquiterpene alcohols, esters, ethers, aldehydes, ketones, oxides and the like. These essential oils and their isolated constituents are frequently utilized as fragrance and flavor agents, and have been widely used in folk medicine for wound healing properties.

Scientific research has corroborated the beneficial effects of essential oils. Essential oils of eucalyptus have been found to “possess central and peripheral analgesic effects as well as neutrophil-dependent and independent anti-inflammatory activities” (Silva et al., 2003, J. Ethnopharmacol. 89(2-3); 277-283), and similar activity has been observed in essential oils from Lavendula angustifolia Mill. (Hajhashemi et al., 2003, J. Ethnopharmacol. 89(1):67-71). Essential oils have been demonstrated to exhibit antibacterial (Bezic et al., 2003, Phytother. Res. 17(9:1037-1040; Goren et al., 2003, Z. Naturforsch. 58(9-10):687-690; de Abreu Gonzaga et al., 2003, Planta Med. 69(8:773-775; Valero and Salmera, 2003, Int. J. Food Microbial. 85(1-2): 73-81) and antifungal (Paranagama et al., 2003, Lett. Appl. Microbiol. 37(1):86-90; Shin, 2003, Arch. Pharm. Res. 26(5):389-393; Velluti et al., 2003, Int. J. Food Microbiol. 89:145-154) activities. Virucidal activity of essential oils has also been observed, including direct virucidal effects against Herpes simplex viruses types 1 and 2 (Garcia et al., Phytother. Res. 17(9):1073-1075; Minami et al., 2003, Microbial Immunol. 47(a):681-684; Schuhmacher et al., 2003, Phytomedicine 10:504-510).

United States Patent Application Publication No. 20050048139 by Modak et al., published Mar. 3, 2005, relates to topical compositions comprising an emollient solvent and an essential oil, which may further comprise additional additives, among which citric acid, glycolic acid and lactic acid are cited.

United States Patent Application Publication No. 20050019431 by Modak et al., published Jan. 27, 2005, relates to compositions comprising a quaternary ammonium compound and an essential oil (or active component thereof).

A number of patent applications relate to compositions comprising an essential oil (or component thereof) where zinc salts are added to inhibit irritation associated with essential oils. Examples of such patent applications include United States Patent Application Publication No. 20040102429 by Modak et al., published May 27, 2004 and United States Patent Application Publication No. 20050238602 by Modak et al., published Oct. 27, 2005, now U.S. Pat. No. 7,435,429.

U.S. Pat. No. 6,858,317 by Aamodt et al., issued Feb. 22, 2005, relates to methods for protecting wood from mold and sap staining fungi which employ a non-toxic mold inhibitor which may be a plant extract such as an essential oil.

U.S. Pat. No. 5,100,652 by Kross et al., issued Mar. 31, 1992, relates to low concentration chlorous-acid generating oral hygiene compositions which may comprise an essential oil as a flavoring agent.

U.S. Pat. No. 5,310,546 by Douglas, issued May 10, 1994, relates to a mouth rinse preparation comprising hydrogen peroxide, zinc chloride, sodium citrate, sodium lauryl sulfate, citric acid and ethanol and optionally an essential oil which is a denaturing agent.

BiON offers several skin care products comprising citric acid, botanicals, and other agents for topical use (San Diego, Calif., US).

Johnson et al. (U.S. Pat. No. 6,319,958 and US20020165130) relates to the use of sesquiterpenoids to promote uptake of exogenous antimicrobial compounds. Similarly, a related article discloses the use of sesquiterpenoids, such as nerolidol, farnesol, bisabolol and apritone, in enhancing bacterial permeability and susceptibility to exogenous antimicrobial compounds, suggesting that sesquiterpenoids have a non-specific and general effect (Brehm-Stecher et al. 2003, Antimicrobial Agents and Chemotherapy, 47(10)3357-3360). In particular, Brehm-Stecher et al. report that nerolidol, farnesol, bisabolol and apritone enhanced the susceptibility of S. aureus to the antibiotics erythromycin, gentamicin, vancomycin, ciproflaxin, clindamycin, and tetracycline.

U.S. Pat. No. 4,867,898 by Spaulding et al., issued Sep. 19, 1989, relates to a liquid hard surface cleaner comprising pine oil and organic, oil-soluble acids at a pH from 0-6.

U.S. Pat. No. 6,753,305 by Raso and Caselli, issued Jun. 22, 2004, relates to a hard surface disinfectant comprising up to 20 percent of cinnamon oil or a component thereof, 0.01-5 percent of an organic acid, and optionally an additional essential oil.

International Patent Application Publication No. WO2007/077573 by Mukhopadhyay, published Jul. 12, 2007, relates to antimicrobial compositions comprising an antimicrobial agent, such as triclosan, and a functionalized hydrocarbon, where the functionalized hydrocarbon can be an essential oil, and/or a solvent.

Infection continues to be the major problem in the management of patients with burn wounds, decubitus ulcers and other surface infections. Control of skin infections is most important in preventing bacteremia and enhancing wound healing. Topical creams containing silver sulfadiazine and other topical antimicrobial agents have been developed and widely used for such purposes. However, complete control of target infection has not been achieved with the use of these agents.

1% silver sulfadiazine (Silvadene®) cream has been effectively used as a prophylactic cream to control burn wound infections. However, it is not very effective in treating established deep wound infections due to the drug's failure to penetrate the wound eschar. The incidence of wound colonization with S. aureus or C. albicans in Silvadene® treated patients has spurred research for other agents.

It has been well established that continuous control of infection facilitates rapid healing of partial thickness burns, decubitus ulcers and other types of surgical wounds and facilitates their closure. Wound healing, especially in burns, is a complex process for which zinc has been found essential. Studies on zinc have shown beneficial results in wound healing with acceleration of the re-epithelialization process and an antibacterial effect. Zinc oxide has been reported to activate endogenous zinc-dependent matrix metalloproteinases, augment expression of endogenous growth factors and facilitate keratinocyte migration.

In earlier studies, topical treatment of burn wounds with zinc sulfadiazine was found to accelerate wound healing better than treatment with silver sulfadiazine. (Gyn and Obstet, 142:553-559 (1976)).

To prevent or reduce infection of burn wounds, topical ointments have been used. These ointments have incorporated silver sulfadiazine (U.S. Pat. No. 3,761,590, incorporated herein by reference) or various antibiotics. A topical ointment for burns has also been reported which contains a combination of silver salts and norfloxocin, a quinoline antibiotic, or its salts (U.S. Pat. No. 4,404,197, incorporated hereby by reference). In the case where the antibiotic is silver norfloxocin, U.S. Pat. No. 4,404,197 reports a synergistic enhancement of activity. U.S. Pat. No. 5,374,432 relates to topical anti-infective ointments containing an antibiotic, silver salt, and sterile carrier. These compositions were found to not only provide improved antimicrobial efficacy, but also reduced incidence of microbial resistance.

U.S. Pat. No. 6,987,133 relates to a topical preparation containing silver sulfadiazine dispersed or solubilized in a cream or lotion base matrix which can be sprayed directly on the burn wound. European Patent No. EP0653214 relates to a topical antibacterial preparation containing silver sulfadiazine and collagen for the treatment of infected hands and for the advancement of their healing.

There is a continuing desire for an antimicrobial or wound healing composition that are non-irritating, safe, and effective for repeated use in various professional and non-professional settings.

### 3. SUMMARY

The compositions of the present subject matter contain low concentrations of benzyl alcohol, one or more essential oil (and/or one or more component (i.e., an “Individual Constituent” or “IC”) thereof) and one or more botanical extract, such as a plant or fruit extract, in combination with one or more alkanediol, one or more solvent, one or more sesquiterpenoids, one or more plant derived alcohol, one or more natural organic acid, natural gelling agents and/or vegetable glycerin. The application is based, at least in part, on the discovery that certain low concentrations of specific combinations of these ingredients have an unexpected synergistic effect, namely the combinations can confer superior antimicrobial properties on personal care, wound healing, veterinary, as well as household products. In certain non-limiting embodiment, the formulations of the present subject matter can be used as a pesticide. Preferably, all components of the preservative composition are derived from a natural (rather than a synthetic) source. Preferably, the compositions of the application have little or no human-discernable fragrance.

In various non-limiting embodiments, the compositions of the present subject matter may include one or more botanical extract, benzyl alcohol, and 1,3-propanediol, wherein the amounts of botanicals and benzyl alcohol are present in a ratio of about 1:1 to 1:12, and wherein the composition pH ranges from 3-5. Optionally, the compositions may further contain fruit acids, additional solvents and/or anti-inflammatory compounds.

In various non-limiting embodiments, the compositions of the present subject matter may be utilized in personal care products such as soaps, scrubs, cosmetics, topical creams and lotions, wound care products, burn wound cream, decubitous ulcer cream (with anti-inflammatory botanicals and the use of silver sulfadiazene as an anti-microbial agent), rapidly acting skin disinfectants, disinfecting wipes, and veterinary products, such as antimicrobial lotion for mastitis, teat dip, and therapeutic ointments. The compositions of the application may be used in concentrations from about 1% to about 10% in personal care products or topical creams.

The present subject matter relates to topical creams containing antimicrobial agents such as silver salts, calendula oil, zinc salts, and curcumin compounds. Non-limiting examples of silver salts include silver sulfadiazine, silver nitrate, silver carbonate, and silver oxide. Additional antimicrobial agents include biguanides (chlorhexidine or polyhexamethelene biguanide), phenoxyethanol, miconazole, polymixin, neomycin bacitracin and povidone These antimicrobial agents provide for the control of infection and promote wound healing in a wide variety of skin lesions, including burns, abrasions, decubitus ulcers, and other local infections.

Furthermore, the synergistic combination of benzyl alcohol and 1,3 propanediol, octanediol and decanediol, which exhibit antifungal activity, is used in the above described topical cream to enhance the antifungal activity. Lactic acid or citric acid is used to assist in the controlled release of silver.

### 4. DETAILED DESCRIPTION

For clarity of description, and not by way of limitation, the detailed description of the application is divided into the following subsections:

(4.1) benzyl alcohol;

(4.2) essential oils;

(4.3) botanical extracts;

(4.4) Sesquiterpenoids;

(4.5) alkanediols;

(4.6) solvents;

(4.7) natural organic acids;

(4.8) combinations of solvents, botanical extracts, and alkanediols;

(4.9) silver and silver salts;

(4.10) zinc and zinc salts;

(4.11) antimicrobials;

(4.12) synergistic combinations of benzyl alcohol and alkanediols;

(4.13) personal care products;

(4.14) wound healing;

(4.15) veterinary products;

(4.16) household/industrial products;

(4.17) medical devices; and

(4.18) pesticides.

In preferred, non-limiting embodiments, the compositions of the application have little or no human-discernable fragrance or scent. While certain embodiments of the application may have a very slight scent, this scent is not sufficient to substantially distort or mask the scent of an added fragrance. Accordingly, the preservative compositions of the application may be used either in unscented products or, alternatively, in products scented with a desired fragrance (for example, a fragrance associated with a particular brand of product). In the latter case, the preservative composition of the application will not substantially alter (or preferably, detectably alter) the character of the desired fragrance. Preferably, the compositions are fragrance-free.

In preferred, non-limiting embodiments of the application, the compositions include botanicals, which include essential oils or individual constituents thereof, and botanical extracts. Each category of botanicals is summarized below.

“About” as used in this document means plus or minus 20 percent of the recited value, so that, for example, “between about 0.125 and 1.0 percent” means a range between 0.125±0.025 and 1.0±0.2.

### 4.1 Benzyl Alcohol

The compositions disclosed herein comprise benzyl alcohol, at a concentration (percent weight/weight, “% w/w”) between about 0.1 and about 10% w/w, or between about 0.1 and about 5% w/w, or between about 0.1 and about 3% w/w, or between about 1.0 and about 3 w/w; or between about 0.5 and about 5% w/w, or between about 0.5 and about 2% w/w. In specific non-limiting embodiments the benzyl alcohol is plant-derived.

In certain, non-limiting embodiments, the compositions disclosed herein comprise benzyl alcohol, at a concentration (percent weight/weight, “% w/w”) between about 0.05 and about 15% w/w; or between about 0.05 and about 10% w/w; or between about 0.05 and about 5% w/w; or between about 0.05 and about 4.5% w/w; or between about 0.05 and about 4% w/w; or between about 0.05 and about 3.5% w/w; or between about 0.05 and about 3% w/w; or between about 0.05 and about 2.5% w/w; or between about 0.05 and about 2% w/w; or between about 0.05 and about 1.5% w/w; or between about 0.05 and about 1% w/w; or between about 0.05 and about 0.5% w/w; or between about 0.05 and about 0.45% w/w; or between about 0.05 and about 0.4% w/w; or between about 0.05 and about 0.35% w/w; or between about 0.05 and about 0.3% w/w; or between about 0.05 and about 0.25% w/w; or between about 0.05 and about 0.2% w/w; or between about 0.05 and about 0.15% w/w; or between about 0.05 and about 0.1% w/vv.

In certain, non-limiting embodiments, the compositions disclosed herein comprise benzyl alcohol, at a concentration of between about 0.1 and about 15% w/w; or between about 0.1 and about 10% w/w; or between about 0.1 and about 5% w/w, between about 0.5 and about 15% w/w; or between about 0.5 and about 10% w/w; or between about 0.5 and about 5% w/w.

In certain, non-limiting embodiments, the compositions disclosed herein comprise benzyl alcohol, at a concentration of between about 0.1 and about 0.5% w/w; or between about 0.1 and about 0.45% w/w; or between about 0.1 and about 0.4% w/w; or between about 0.1 and about 0.35% w/w; or between about 0.1 and about 0.3% w/w; or between about 0.1 and about 0.25% w/w; or between about 0.1 and about 0.2% w/w; or between about 0.1 and about 0.15% w/w.

In certain, non-limiting embodiments, the compositions disclosed herein comprise benzyl alcohol present in stock solutions in amounts ranging from between about 1 and about 80% w/w; or between about 1 and about 70% w/w; or between about 1 and about 60% w/w, or between about 1 and about 50% w/w; or between about 1 and about 40% w/w.

### 4.2 Essential Oils

Essential oils (“EOs”), as defined herein, are volatile oils obtained from plant or animal sources, or their synthetic equivalents, and are composed of complex mixtures of several constituents such as monoterpenes and sesquiterpene hydrocarbons, monoterpene and sesquiterpene alcohols, esters, ethers, aldehydes, ketones, oxides and the like. Examples of EOs include, but are not limited to, cinnamon oil, basil oil, bergamot oil, clary sage oil, ylang-ylang oil, neroli oil, sandalwood oil, frankincense oil, ginger oil, peppermint oil, lavender oil, jasmine absolute, geranium oil bourbon, spearmint oil, clove oil, patchouli oil, rosemary oil, rosewood oil, sandalwood oil, tea tree oil, vanilla oil, lemongrass oil, oregano oil, thymol, galanga oil, cedarwood oil, balsam oils, tangerine oil, Hinoki oil, Hiba oil, ginko oil, eucalyptus oil, lemon oil, orange oil, sweet orange oil, pomegranate seed oil, pomegranate oil, manuka oil, citronella oil, curry leaf oil, and calendula oil.

Individual constituents (“ICs”) of essential oils may be isolated from the oil (natural) or may be entirely or partially chemically synthetic, and include, but are not limited to, thyme, oregano, curcumin, 1-citronellol, α-amylcinnamaldehyde, lyral, geraniol, farnesol, hydroxycitronellal, isoeugenol, eugenol, camphor, eucalyptol, linalool, citral, thymol, limonene and menthol. Further examples of ICs include sesquiterpenoid compounds, which may be the active compounds in the essential oils. Sesquiterpenoid compounds, containing 15 carbons, are formed biosynthetically from three 5-carbon isoprene units. Sesquiterpenoid compounds include, but are not limited to, farnesol, nerolidol, bisabolol, apritone, chamazulene, santalol, zingiberol, carotol, and caryophyllen.

Mixtures of one or more EO, one or more IC, and one or more EU as well as one or more IC, are encompassed by the present subject matter. In specific non-limiting embodiments of the application, an IC is selected from the (non-limiting) group consisting of camphor, curcumin, alpha-pinene, constituents of cinnamon leaf oil such as, cinnamaldehyde, cinnamylacetic ester, cinnamic acid, ethyl cinnamate, methyl chavicol, linalool, beta-caryophyllene, and eugenol; constituents of lemongrass oil such as d-limonene, geranyl acetate, nerol, geraniol, citral, and/or myrcene; constituents of citronella oil such as geraniol, citronellol, citronellal, geranyl acetate, limonene, methyl isoueugenol, and/or elemol; components of basil oil such as camphor, limonene, and/or β-selinene; and constituents of orange oil such as α-pinene, sabinene, myrcene, limonene, linalool, citronellal, neral and/or geranial. An EO or IC for use in the application may be obtained from its natural source or may be chemically synthesized.

In preferred non-limiting embodiments of the application, the EO is selected from one or more EO from the group consisting of cinnamon oil (CO) (bark or leaf), lemongrass oil (LGO), and basil oil (BO), all of which have little to no fragrance, or nonfragrant oils such as pomegranate seed oil (PSO).

Calendula contains high amounts of flavonoids, plant-based antioxidants that protect the body against cell-damaging free radicals. It appears to have anti-inflammatory, antiviral, and antibacterial effects. Animal studies show that calendula accelerates wound healing, possibly by increasing blood flow to the wounded area and by helping the body produce collagen proteins, which are used to heal skin and connective tissue.

In various non-limiting embodiments, low concentrations of essential oils and ICs are used. Essential oils or ICs are present in stock solutions in amounts ranging from about 0.05% to about 30% (w/w). In alternative embodiments, for example compositions that may be used without dilution, the amounts range from about 0.01% to about 1% (w/w). In alternative embodiments, for example compositions that may be used without dilution, the amounts range from about 0.01% to about 5% (w/w), or from about 0.01% to about 4% (w/w), or from about 0.01% to about 3% (w/w), or from about 0.01% to about 2% (w/w), or from about 0.01% to about 1% (w/w), or from about 0.02% to about 5% (w/w), or from about 0.02% to about 4% (w/w), or from about 0.02% to about 3% (w/w), or from about 0.02% to about 2% (w/w), or from about 0.02% to about 1% (w/w). These concentrations (and others recited throughout) may be increased in stock solutions intended for dilution.

In specific non-limiting embodiments of the application, an IC is selected from the (non-limiting) group consisting of a curcumin compound and calendula oil. In various non-limiting embodiments, low concentrations of essential oils and ICs are used. Specifically, calendula oil are used in amounts ranging from about 0.3 to about 5% w/w, and curcumin compounds are used in amounts ranging from about 0.02 to about 0.2% w/w. These concentrations (and others recited throughout) may be increased in stock solutions intended for dilution.

### 4.3 Botanical Extracts

Botanical extracts, as defined herein, include plant, herbal, and fruit extracts, which are not “essential oils” as noted above. The botanical extracts utilized herein include but are not limited to wasabi, honey suckle, cedarwood, aspen bark, willow bark, Camellia sinensis (green tea), grapes, pomegranate, Echinacea, Centella Asiatica, Elderflower, Irish moss, Mallow, soap bark, Yucca, Clary sage, oregano, thyme, curcumin compounds, resveratrol (polyphenolic compound from grape, berries, etc.) vetivert and mixtures thereof. The botanical utilized to obtain the botanical extract may be obtained from any of the plant parts including the leaves, pulp, seeds, or stems, fruit and fruit seeds, as well as the whole plant. Herbal extracts can be, for example, standardized extracts that are dispersible and/or soluble in aqueous medium.

Examples of herbal extracts include, without limitation, extracts of chamomile, rosemary, aloe, nettle, Centella asiatica, ginkgo biloba, betula, and witch hazel. Such extracts may be delivered in a carrier such as water, propylene glycol, hydroalcohol, glycerine, or butylene glycol. Additional extracts with nutritional quality can be used, including, without limitation, green tea, white tea, grape skin, grape seed, grapefruit, grapefruit seed, grapefruit peel, citrus fruits (other than grapefruit extract) bilberry, blueberry, Ginkgo biloba, soy isoflavones, soy extract, fermented soy protein, black cohosh, St. John's wort, echinacea, chamomile, rosemary, aloe extract and juice, nettle, coconut fruit and Centella asiatica. Botanical extracts can be obtained from, for example, Active Organics (Lewisville, Tex.), New Age Botanicals (Garland, Tex.), Triarco Industries (Wayne, N.J.), and Aloecorp (Broomfield, Colo.).

Examples of nonfragrant botanicals include pomegranate seed oil (PSO), mixtures of edible plant extract Kefiprotect (KP), and tetrahydrocurcuminoid (THC). Turmeric and curcuminoids have been documented to have anti inflammatory, antioxidant and wound healing properties. The following curcuminoids can be used in topical creams, tetrahydrocurcumin, tetrahydrodemethoxycurcumin, tetrahydrobisdemethoxycurcumin, and mixtures thereof. Additional examples of botanical extracts include coconut derived phospholipid (Arlasik phospholipid PTM), natural blends of fatty acids which mimic those found in the stratum corneum, mixture of fatty acids with pigments such as carotenes, carotenoids or phytosterols that are known to facilitate repair to damaged skin, and the like. Specific examples of useful botanical extracts include avocado, which contains the sterol sitosterol; carrot, which contains beta carotene; sesame oil which contains a mixture of saturated and unsaturated fatty acids, and brazil nut oil. Because of its broad distribution of fatty acids, extracts such as brazil nut oil, can outperform single fatty acids with respect to incorporation into the lipid lamellar structures. Brazil nut oil (BNO) originates from the harvested fruit from the South American rain forest tree: Bertholletia excelsa.

Botanical extracts also include flavonoids and terpenoids. The flavonoids contemplated by the present subject matter include, but are not limited to, turin, quercetin, hesperidin, and naringin. Terpenoids contemplated by the present subject matter include, but are not limited to, monoterpenes, sesquiterpenes, and diterpenes.

In preferred non-limiting embodiments of the application, the botanical extract is selected from one or more extract selected from the group consisting of grapefruit seed extract (GSE), pomegranate seed oil (PSO), citrus fruit extract, or mixtures of edible plant extract Kefiprotect (KP), coconut derived phospholipid (Arlasik phospholipid PTM), and tetrahydrocurcuminoid (THC).

In various non-limiting embodiments, low concentrations of botanical extracts are used. Botanical extracts are present in stock solutions or compositions for use in concentrations ranging from about 2.0% to about 45% (w/w), or from about 10% to about 20% (w/w). In alternative embodiments, the concentrations range from about 0% to about 20% (w/w), or from about 0% to about 10% (w/w), or from about 0.5% to about 10% (w/w), or from about 0% to about 4% (w/w). Alternative embodiments use from about 5% to about 10% (w/w).

In alternative embodiments the amounts range from about 0.01% to about 5% (w/w), or from about 0.01% to about 4% (w/w), or from about 0.01% to about 3% (w/w), or from about 0.01% to about 2% (w/w), or from about 0.01% to about 1% (w/w), or from about 0.01% to about 0.5% (w/w), or from about 0.04% to about 0.5% (w/w).

In alternative embodiments the amounts range from about 0.1% to about 5% (w/w), or from about 0.1% to about 4% (w/w), or from about 0.1% to about 3% (w/w), or from about 0.1% to about 2% (w/w), or from about 0.1% to about 1% (w/w), or from about 0.2% to about 4% (w/w), or from about 0.2% to about 1% (w/w).

In alternative embodiments, the amounts range from about 0.5% to about 20% (w/w), or from about 0.5% to about 15% (w/w), or from about 0.5% to about 10% (w/w), or from about 0.5% to about 5% (w/w).

### 4.4 Sesquiterpenoids

Compositions disclosed herein comprise one or more sesquiterpenoid selected from the group consisting of farnesol, nerolidol, bisabolol, apritone, citral and combinations thereof. Where present, the sesquiterpenoid is at a concentration between about 0.1 and about 4% w/w, or between about 0.1 and 3% w/w, or between about 0.3 and about 3% w/w, or between about 1 and 3% w/w, or between about 0.1 and 0.3% w/w; or between about 0.5 and about 4% w/w, or between about 0.5 and 3% w/w; or between about 0.5 and 0.3% w/w, or between about 0.5 and 2% w/w.

### 4.5 Alkanediols

In non-limiting embodiments, bifunctional alcohols which may be used according to the present subject matter are alkanediols. Suitable alkanediols include, but are not limited to, propanediol, butanediol, dodecanediol, decanediol, nonanediol, octanediol, heptanediol, hexanediol, and pentanediol.

In particular non-limiting embodiments, the alkanediols have a carbon backbone of between 3 and 25 carbon atoms, including but not limited to 1,9 Nonanediol, 1,2-Decanediol, 1,10-Decanediol, 1,11-Undecanediol, 1,2-Dodecanediol, 1,12 Dodecanediol, Cyclododecanediol, 1,13-Tridecanediol, 1,2-Tetradecanediol, 1,14-Tetradecanediol, 1,15-Pentadecanediol, 1,16-Hexadecanediol, 1,17-Heptadecanediol, 1,18-Octadecanediol, 1,19-Nonadecanediol, 1,20-Eicosanediol, 1,21-Heneicosanediol, 1,22-Docosanediol, 1,23-Tricosanediol, 1,24-Tetracosanediol, 1,25-Pentacosanediol. A preferred non-limiting alkanediol is 1,3 propanediol (Zemea®), which is a natural product prepared from corn sugar.

In non-limiting embodiments of the application, the stock solution concentration of the alkanediols ranges from about 0.5% to about 70% (w/w), or from about 10% to about 70% (w/w). In alternative embodiments, the concentration of alkanediols ranges from about 0% to about 50% (w/w), or from about 0% to about 10% (w/w), or from about 5% to about 10% (w/w). In other embodiments, the concentration of alkanediols ranges from about 1% to about 20% (w/w), or from about 1.5% to about 15% (w/w), or from about 2% to about 10% (w/w), or from about 2.5% to about 10% (w/w), or from about 3% to about 10% (w/w). In other embodiments, the concentration of alkanediols ranges from about 1% to about 70% (w/w), or from about 1% to about 60% (w/w), 1% to about 50% (w/w), or from about 1% to about 40% (w/w).

In certain non-limiting embodiments of the application, the concentration of the alkanediols present in a composition for use ranges from about 0.1% to about 15% (w/w), or from about 0.1% to about 10% (w/w), or from about 0.1% to about 5% (w/w), or from about 0.3% to about 5% (w/w), or from about 0.5% to about 2% (w/w).

### 4.6 Solvents

In various non-limiting embodiments, the compositions of the present subject matter may include one or more solvent, including but not limited to solvent(s) selected from the group consisting of water, alcohols, plant derived alcohols, glycols, glycerol, glycerine, octoxyglycerin, diglycerol, propylene glycol, dipropylene glycol, and vegetable oils.

Preferred but non-limiting examples of non-alkanediol alcohols for solubilization are aliphatic alcohols having between about 1 and 8 carbon atoms such as methanol, ethanol, n-propanol, isopropyl alcohol, 2-methyl-2 propanol, hexanol, or combinations thereof. Aromatic alcohols, for example, but not by way of limitation, phenoxyethanol, benzyl alcohol, 1-phenoxy-2-propanol, and/or phenethyl alcohol, may also optionally be used in combination with aliphatic alcohols.

Aromatic alcohols, for example, but not by way of limitation, include phenoxyethanol, benzyl alcohol, 1-phenoxy-2-propanol, and/or phenethyl alcohol, for example at a concentration of between about 0.5 and 5% (weight/weight) may also optionally be used in combination with aliphatic alcohols. A further solvent which optionally may be comprised in a composition of the application is isopropyl myristate. Additional aliphatic alcohols include ethanol, denatured alcohol (SDA 40B and SDA 3C) and isopropanol.

Compositions comprising synergistic combination of benzyl alcohol, botanicals, and 1,3 propanediol and its derivatives such as 2-methyl-1-nitro 1,3-propanediol (Diol) or 2-Hydroxymethyl 2-nitro 1,3-propanediol (Triol), further contain cosolvents such as glycerin, octoxyglycerin, alcohol, glycols, butanediol, and phenoxy ethanol.

In preferred non-limiting embodiments of the application, the solvent is benzyl alcohol, glycerin, or a combination thereof.

In certain embodiments, the solvents are used in stock solution, or in a composition for use, at concentrations ranging from about 0% to about 90% (w/w), or from about 0% to about 85% (w/w), or from about 0% to about 70% (w/w), or from about 30% to about 65% (w/w). In certain embodiments, the solvents are present in a composition for use at a concentration of between about 1% to about 50% (w/w), or from about 2% to about 40% (w/w), or from about 5% to about 70% (w/w), or from about 5% to about 50% (w/w), or from about 5% to about 20% (w/w). Benzyl alcohol concentrations range from about 0% to about 90%, more preferably from about 0% to about 70%, preferably from about 5% to about 90% (w/w). In other embodiments, the concentrations are from about 1% to about 10% (w/w), more preferably from about 5% to about 10% (w/w). In alternative embodiments, the concentration ranges range from about 5% to about 90% (w/w), preferably from about 30% to about 90% (w/w), and more preferably from about 40% to about 80% (w/w). In a preferred embodiment, the solvent is a natural product, for example, benzyl alcohol derived from the Cassia plant.

In alternative preferred non-limiting embodiments of the application, the solvent is benzyl alcohol or its derivatives, e.g., hydroxyl benzyl alcohol, nitro benzyl alcohol, or other derivatives. Benzyl alcohol concentrations ranging from about 0.5% to about 10% (w/w), preferably from about 0.5% to about 5% (w/w), more preferably from about 0.5% to about 4% (w/w), have been found to exhibit synergistic antimicrobial efficacy with certain botanical organic acids, and in particular fruit acids. Alternative embodiments use from about 1.0% to about 5.0% (w/w), or from about 1% to about 3% (w/w) benzyl alcohol. Use of other botanicals and synthetic antimicrobials along with benzyl alcohol and these acids further enhances the synergistic activity as discussed in further detail below.

### 4.7 Natural Organic Acids

Natural organic acids, such as fruit acids, which may be used according to the application include but are not limited to fruit acid, citric acid, glycolic acid, lactic acid, malic acid, tartaric acid, benzoic acid, salicylic acid, and acetic acid. In certain non-limiting embodiments, the fruit acid is Multifruit BSC (Arch Chemicals), which is a mixture of lactic, citric, tartaric, glycolic, and malic acid extracted from plants. In preferred non-limiting embodiments of the application, the fruit acid is lactic acid. A fruit acid for use in the application may be obtained from its natural source or may be chemically synthesized.

Organic acids may also be used according to the application. Organic acids include but are not limited to benzoic acid and its derivatives including salt forms, for example, a benzyl benzoate, paramino benzoic acid, nitro benzoic acid, hydroxyl benzoic acid, fluorobenzoic acid, and benzyl salicylate.

Fruit acids may be used according to the application to assist in the controlled release of the silver compound. Non-limiting examples of fruit acids include but are not limited to citric acid, glycolic acid, lactic acid, malic acid, tartaric acid and acetic acid. In certain non-limiting embodiments, the fruit acid is Multifruit BSC (Arch Chemicals), which is a mixture of lactic, citric, tartaric, glycolic, and malic acid extracted from plants. A fruit acid for use in the compositions of the present subject matter may be obtained from its natural source or may be chemically synthesized. In preferred non-limiting embodiments of the application, the fruit acid is lactic acid or citric acid.

In non-limiting embodiments of the application, the stock solution concentrations of the fruit acids ranges from about 0% to about 70% (w/w), or from about 5% to about 70% (w/w), or from about 5% to about 20% (w/w), or from about 10% to about 20% (w/w). In alternative non-limiting embodiments of the application, the concentrations range from about 0% to about 20% (w/w), or from about 0% to about 40% (w/w), or from about 0.1% to about 20% (w/w), or from about 0.2% to about 4% (w/w), or from about 0.5% to about 4% (w/w), or from about 2% to about 4% (w/w).

In alternative embodiments, the concentrations of natural organic acid present in a composition for use range from about 0.2% to about 2% (w/w), or from about 0.2 to about 1% (w/w). In another alternative embodiments, the natural organic acids are present in a composition for use at concentrations ranging from between about 0.01% to about 5% (w/w), or from about 0.01% to about 4% (w/w), or from about 0.01% to about 3% (w/w), or from about 0.01% to about 2% (w/w), or from about 0.01% to about 1% (w/w), or from about 0.02% to about 5% (w/w), or from about 0.02% to about 4% (w/w), or from about 0.02% to about 3% (w/w), or from about 0.02% to about 2% (w/w), or from about 0.02% to about 1% (w/w), or from about 0.05% to about 2.0% (w/w), or from about 0.1% to about 2.0% (w/w).

### 4.8 Combinations of Solvents, Botanical Extracts, and Alkanediols

In various non-limiting embodiments, the present subject matter provides for compositions comprising a combination of a solvent, a botanical extract, and an alkanediol. Preferably, this combination produces a synergistic anti-microbial effect against at least one microbe selected from the group consisting of Escherichia coli, Pseudomonas aeruginosa, Staphylococcus aureus, methicillin-resistant S. aureus, and Candida albicans (“synergistic” means that the antimicrobial effect of the combination is greater than the sum of the antimicrobial effects of the individual components).

In particular, non-limiting embodiments, the present subject matter provides for formulations that are concentrated and may be diluted to provide a composition for personal, household, or industrial use. The present subject matter further provides for methods of providing an antimicrobial effect to a surface comprising applying, to the surface, an effective amount of a composition as described herein. An antimicrobial effect means killing and/or inhibiting the growth/proliferation of a microbe. In particular non-limiting embodiments of the application, the microbe is selected from the group consisting of from the group consisting of Escherichia coli, Pseudomonas aeruginosa, Staphylococcus aureus, methicillin-resistant S. aureus, and Candida albicans. In specific non-limiting embodiments, the composition is exposed to the surface for at least 20 seconds, at least 30 seconds, or at least 60 seconds, or at least 5 minutes or at least 10 minutes. In various non-limiting embodiments, the surface may be the a skin or mucosal surface, a household surface (e.g., a surface of a countertop, table sink, toilet, wall, floor, appliance, window, shower surface, rug, upholstery, fabric, etc.) or an industrial surface (e.g., a surface of a countertop, table sink, toilet, wall, floor, appliance, window, shower surface, rug, upholstery, fabric, etc.).

In particular, non-limiting embodiments of the application, the compositions comprise between about 0.0 and 70% (w/w) of one or more solvent, between 10% and 70% (w/w) alkanediols, and between about 2.0 and 45% (w/w) essential oils and/or botanical extracts. In a particular embodiment of the application, the compositions comprise benzyl alcohol, 1,3 propanediol (Zemea®), and grapefruit seed extract (GSE). Fragrance free botanicals such as grape fruit seed extract (GSE), Curcumin compounds (CR.MN) with or without fruit acid exhibits synergistic antimicrobial efficacy with benzyl alcohol. The stability and efficacy of the composition can be enhanced by the use of 1,3 propanediol.

In certain non-limiting examples of the application, the antimicrobial composition contains synergistic amounts of benzyl alcohol and botanicals. In these embodiments, the inclusion of an alkanediol, such as 1,3-propanediol, which acts as an emollient solvent, is optional. The benzyl alcohol has been found to exhibit synergistic antimicrobial efficacy with certain botanical organic acids, in particular fruit acids. Use of other botanicals and synthetic antimicrobials along with benzyl alcohol and these acids further enhances the synergistic activity. A nonlimiting example of an antimicrobial composition containing the synergistic combination includes from about 0.5 to about 10% (w/w) benzyl alcohol and from about 0.2 to about 4% (w/w) fruit acids which include but are not limited to lactic acid, citric acid, plant-based benzoic acid, and combinations thereof. These compositions exhibit broad spectrum and persistent activity at a pH range from about 3.0 to about 6.0, preferably from about 3.0 to about 5.0.

In addition to the above ingredients, a composition of the application may optionally further comprise natural gelling agents, vegetable glycerin, plant derived alcohol, an emollient to further reduce irritation, such as, but not limited to, a fatty alcohol, behentrimonium methosulfate-cetyl alcohol (Incroquat TMS), or a polyol such as glycerol, propylene glycol, diglycerol, ethylene glycol, diethylene glycol, triethylene glycol, dipropylene glycol, tripropylene glycol, hexylene glycol, butylene glycol, etc.

Essential oils are volatile and therefore it is desirable that the antimicrobial composition containing essential oils is incorporated in a suitable base in which it is stable at higher temperature and over a long period of time. Accordingly, a composition of the application may optionally comprise a hydrophilic or hydrophobic gel forming polymer, a fatty acid, a plant oil, etc. Suitable hydrophilic gel polymers include, but are not limited to, hydroxypropylmethyl cellulose, cationic hydroxyethyl cellulose (U-care polymers), ethyl cellulose, hydroxypropyl cellulose, hydroxymethyl cellulose, carboxy methyl cellulose, polyethylene oxide (polyox resins), and chitosan pyrrolidone carboxylate (Kytamer PC), silica gel, carbomerpolymers etc. Suitable hydrophobic gel polymers include, but are not limited to, silicone polymers, for example polydimethylsiloxane polymer (Dow Corning 225 Silicone Fluid), dimethiconol fluid in dimethicone (Dow Corning 1403 Silicone Fluid), cyclomethicone and dimethicone copolyl (Dow Corning 3225C and Q2-5220 Silicone Fluid), silicone glycol (BASF 1066 DCG polyol), KSG series Silicone gels (Shin-etsu), and combinations thereof. Suitable plant oils include, but are not limited to, olive oil, almond oil, avocado oil, basil oil, primrose oil, peanut oil, safflower oil, sesame oil, soya or soy bean oil, wheat germ oil.

The compositions of the present subject matter may optionally further contain other botanicals or synthetic antimicrobial compounds. Exemplary but non-limiting antimicrobials may include synthetic antimicrobial agents such as quaternary ammonium compounds such as benzalkonium chloride and/or benzethonium chloride, biguanides, chlorhexidine, polyhydroxymethylbiguanide (PHMB), Vantocil (polyiminoimidocarbonyliminoimidocarbonyl-iminohexamethylene) hydro-chloride, chlorinated phenols (Triclosan, PCMX (Para Chloro Meta Xylenol)), propanediol and their derivatives, iodine compounds, silver salts, and antifungal agents such as miconazole. Concentrations range from about 0% to about 10% (w/w), preferably from about 0% to about 5% (w/w), more preferably from about 0% to about 2% (w/w).

### 4.9 Silver and Silver Salts

The silver component of the application may be elemental silver or a silver salt. Suitable silver salts include silver acetate, silver benzoate, silver carbonate, silver iodate, silver iodide, silver lactate, silver laurate, silver nitrate, silver oxide, silver palmitate, silver protein and silver sulfadiazine.

Non-limiting examples of silver salts include silver sulfadiazine in an amount ranging from about 0.5 to about 1% w/w, silver nitrate in an amount ranging from about 0.2 to about 0.5% w/w, silver carbonate in an amount ranging from about 0.2 to about 0.5% w/w, and silver oxide in an amount ranging from about 0.2 to about 0.5% w/w. The preferred compound for use as the silver component is silver sulfadiazine (AgSD).

### 4.10 Zinc and Zinc Salts

Suitable zinc salts for use in these formulations include zinc acetate (molar solubility in water of 1.64 moles/1), zinc butyrate (molar solubility in water of 0.4 moles/1), zinc citrate (molar solubility in water of <0.1 moles/1), zinc gluconate (molar solubility in water of 0.28 moles/1), zinc glycerate (moderately water soluble), zinc glycolate (moderately water soluble), zinc formate (molar solubility in water of 0.33 moles/1), zinc lactate (molar solubility in water of 0.17 moles/1), zinc picolinate (moderately water soluble), zinc propionate (molar solubility in water of 1.51 moles/1), zinc salicylate (low water solubility), zinc tartrate (moderately water soluble) and zinc undecylenate (moderately water soluble).

Combinations of zinc salts may be used, as soluble and nonsoluble salts. Zinc salts are used in amounts ranging from about 0.2 to about 1% w/w.

### 4.11 Antimicrobials

Various embodiments of the application may comprise one or more antimicrobial agent. Non-limiting examples of antimicrobial agents include, but are not limited to, chlorhexidine gluconate (CHG), benzalkonium chloride (BZK), or iodopropynylbutyl carbamate (IPBC; Germall plus). Further examples of antimicrobial agents include, but are not limited to, iodophors, iodine, benzoic acid, dihydroacetic acid, propionic acid, sorbic acid, methyl paraben, ethyl paraben, propyl paraben, butyl paraben, cetrimide, quaternary ammonium compounds, including but not limited to benzethonium chloride (BZT), dequalinium chloride, biguanides such as chlorhexidine (including free base and salts (see below)), PHMB (polyhexamethylene biguanide), chloroeresol, chlorxylenol, benzyl alcohol, bronopol, chlorbutanol, ethanol, phenoxyethanol, phenylethyl alcohol, 2,4-dichlorobenzyl alcohol, thiomersal, clindamycin, erythromycin, benzoyl peroxide, mupirocin, bacitracin, polymyxin B, neomycin, triclosan, parachlorometaxylene, foscarnet, miconazole, fluconazole, itriconazole, ketoconazole, and pharmaceutically acceptable salts thereof. Additional antimicrobial agents may be used in the present compositions.

Pharmaceutically acceptable chlorhexidine salts that may be used as antimicrobial agents according to the application include, but are not limited to, chlorhexidine palmitate, chlorhexidine diphosphanilate, chlorhexidine digluconate, chlorhexidine diacetate, chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluorophosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine malate, chlorhexidine tartrate, chlorhexidine dimonoglycolate, chlorhexidine monodiglycolate, chlorhexidine dilactate, chlorhexidine di-.alpha.-hydroxyisobutyrate, chlorhexidine diglucoheptonate, chlorhexidine di-isethionate, chlorhexidine dibenzoate, chlorhexidine dicinnamate, chlorhexidine dimandelate, chlorhexidine di-isophthalate, chlorhexidine di-2-hydroxynapthoate, and chlorhexidine embonate. Chlorhexidine free base is a further example of an antimicrobial agent. These and further examples of antimicrobial agents useful in the compositions of this application can be found in such references as Goodman and Gilman's The Pharmacological Basis of Therapeutics (Goodman Gilman A, Rall T W, Nies A S, Taylor P, ed. (Pergamon Press; Elmsford, N.Y.: 1990)), the contents of which are hereby incorporated by reference.

In preferred embodiments of the application, the antimicrobials include biguanides (chlorhexidine or polyhexamethelene biguanide), phenoxyethanol, miconazole, polymixin, neomycin, bacitracin and povidone iodine. Such antimicrobials are used in amounts ranging from about 0.1 to about 2.0% w/w.

### 4.12 Synergistic Combinations of Benzyl Alcohol and Alkanediols

In non-limiting embodiments, bifunctional alcohols which may be used according to the present subject matter are alkanediols. Suitable alkanediols include, but are not limited to, 1,3 propanediol, dodecanediol, decanediol, nonanediol, octanediol, heptanediol, hexanediol and pentanediol. In particular non-limiting embodiments, the alkanediols have a carbon backbone of between 3 and 25 carbon atoms, including but not limited to 1,9 Nonanediol, 1,2-Decanediol, 1,10-Decanediol, 1,11-Undecanediol, 1,2-Dodecanediol, 1,12 Dodecanediol, Cyclododecanediol, 1,13-Tridecanediol, 1,2-Tetradecanediol, 1,14-Tetradecanediol, 1,15-Pentadecanediol, 1,16-Hexadecanediol, 1,17-Heptadecanediol, 1,18-Octadecanediol, 1,19-Nonadecanediol, 1,20-Eicosanediol, 1,21-Heneicosanediol, 1,22-Docosanediol, 1,23-Tricosanediol, 1,24-Tetracosanediol, 1,25-Pentacosanediol.

In a preferred non-limiting embodiment, the alkanediol is 1,3 propanediol (Zemea®), which is a natural product prepared from corn sugar. In another non-limiting embodiment, the alkanediols include 1,3 propanediol, octanediol and decanediol, and mixtures thereof. The alkanediols are present in amounts ranging from about 0.2 to about 1% w/w. In preferred embodiments the benzyl alcohol may be prepared from a natural source such as the Casia plant.

In various non-limiting embodiments, the compositions of the present subject matter may include a solvent including but not limited to water, alcohols, glycols, glycerol, glycerine, octoxyglycerine, diglycerol, propylene glycol, dipropylene glycol, and vegetable oils. Non-limiting examples of non-alkanediol alcohols for solubilization are aliphatic alcohols having carbon atoms about 1 to 8 such as methanol, ethanol, n-propanol, isopropyl alcohol, 2-methyl-2-propanol, hexanol, or combinations thereof. Aromatic alcohols, for example, but not by way of limitation, phenoxyethanol, benzyl alcohol, 1-phenoxy-2-propanol, and/or phenethyl alcohol, may also optionally be used in combination with aliphatic alcohols, aromatic alcohols, for example, but not by way of limitation, include phenoxyethanol, benzyl alcohol, 1-phenoxy-2-propanol, and/or phenethyl alcohol, for example at a concentration of between about 0.5 and 5 percent (weight/weight) may also optionally be used in combination with aliphatic alcohols. A further solvent which optionally may be comprised in a composition of the application is iso propyl myristate. Additional aliphatic alcohols include ethanol, denatured alcohol (SDA 40B and SDA 3C) and isopropanol. A preferred non-limiting solvent is benzyl alcohol, which is used in amounts ranging from about 0.5 to about 5% w/w.

### 4.13 Personal Care Products

The compositions of the application may be used as alternatives to conventional preservatives or may be combined with one or more antimicrobial agent to enhance their activity, particularly providing persistent antimicrobial protection without causing skin sensitivity.

In non-limiting embodiments, the present subject matter provides for personal care product compositions comprising low concentrations of one or more essential oil and/or one or more botanical extract, for example a plant or fruit extract, in combination with one or more solvent and one or more alkanediol. In preferred, non-limiting embodiments, the above-listed components produce a synergistic antimicrobial effect, and the low concentrations of the active agents are such that regular exposure of skin to the personal care product does not produce skin irritation in a normal subject. Preferably, the pH of personal care products is between about 3.0 and 6.0.

Non-limiting examples of personal care products which may utilize the compositions of the present subject matter include bar soap, liquid soap (e.g., hand soap), hand sanitizer (including rinse off and leave-on alcohol based and aqueous-based hand disinfectants), preoperative skin disinfectant, cleansing wipes, disinfecting wipes, body wash, acne treatment products, antifungal diaper rash cream, antifungal skin cream, shampoo, conditioner, cosmetics (including but not limited to liquid or powder foundation, liquid or solid eyeliner, mascara, cream eye shadow, tinted powder, “pancake” type powder to be used dry or moistened, etc.) deodorant, antimicrobial creams, body lotion, hand cream, topical cream, aftershave lotion, skin toner, mouth wash, toothpaste, sunscreen lotion, and baby products such as, but not limited to, cleansing wipes, baby shampoo, baby soap, and diaper cream. The present subject matter may also be applied to wound care items, such as, but not limited to, wound healing ointments, creams, and lotions, wound coverings, burn wound cream, bandages, tape, and steri-strips, and medical articles such as medical gowns, caps, face masks, and shoe-covers, surgical drops, etc. Additional products include but are not limited to oral products such as mouth rinse, toothpaste, and dental floss coatings, veterinary and pet care products, preservative compositions, and surface disinfectants including solutions, sprays or wipes.

Personal care compositions according to the application, in addition to botanical extract, solvent, and alkanediol, may further comprise one or (preferably) more than one component selected from the group consisting of emollients, stabilizing agents, thickening agents, humectants, anti-inflammatory agents, antimicrobial agents, neutralizing agents, surfactants, water, silicone polymers, alcohols, and hydrogels, as well as additional components as may be known in the art. Non-limiting examples of such components are set forth below.

In various non-limiting embodiments, a personal care product comprising a combination of one or more essential oil and/or IC together with one or more fruit acid may further comprise an emollient, for example PEG 20 almond glycerides, Probutyl DB-10, Glucam P-20, Glucam E-10, Glucam P-10, Glucam E-20, Glucam P-20 distearate, glycerin, propylene glycol, octoxyglycerin, cetyl acetate, acetylated lanolin alcohol (e.g., Acetulan), cetyl ether (e.g., PPG-10), myristyril ether (e.g., PPG-3), hydroxylated milk glycerides (e.g., Cremeral HMG), polyquaternium compounds (e.g., U-care compounds), copolymers of dimethyl dialyl ammonium chloride and acrylic acid (e.g., Merquat), dipropylene glycol methyl ethers (e.g., Dowanol DPM, Dow Corning), polypropylene glycol ethers (e.g., Ucon 50-HB-600, Union Carbide) and silicon polymers. Other suitable emollients may include hydrocarbon-based emollients such as petrolatum or mineral oil, fatty ester-based emollients, such as methyl, isopropyl and butyl esters of fatty acids such as isopropyl palmitate, isopropyl myristate, isopropyl isostearate, isostearyl isostearate, diisopropyl sebacate, and propylene dipelargonate, 2-ethylhexyl isononoate, 2-ethylhexyl stearate, C₁₂-C₁₆ fatty alcohol lactates such as cetyl lactate and lauryl lactate, isopropyl lanolate, 2-ethylhexyl salicylate, cetyl myristate, oleyl myristate, oleyl stearate, oleyl oleate, hexyl laurate, and isohexyl laurate. Additional useful emollients include lanolin, olive oil, cocoa butter, and shea butter.

In various non-limiting embodiments, a personal care product comprising a combination of one or more essential oil and/or IC together with one or more fruit acid may further comprise a stabilizing agent consisting of antioxidants, including but not limited to vitamin C (ascorbic acid) and vitamin E (tocopherol), and surfactants, including but not limited to incromide or silicone-based surfactants (Masil SF-19, BASF).

In various non-limiting embodiments, a personal care product comprising a combination of one or more essential oil and/or IC together with one or more fruit acid may further comprise a thickening and/or gelling agent such as stearyl alcohol, cationic hydroxy ethyl cellulose (Ucare; JR30), hydroxy propyl methyl cellulose, hydroxy propyl cellulose (Klucel), chitosan pyrrolidone carboxylate (Kytamer), behenyl alcohol, zinc stearate, emulsifying waxes, including but not limited to Incroquat and Polawax, an addition polymer of acrylic acid, a resin such as Carbopol® ETD™ 2020, guar gum, acacia, acrylates/steareth-20 methacrylate copolymer, agar, algin, alginic acid, ammonium acrylate co-polymers, ammonium alginate, ammonium chloride, ammonium sulfate, amylopectin, attapulgite, bentonite, C9-15 alcohols, calcium acetate, calcium alginate, calcium carrageenan, calcium chloride, caprylic alcohol, carbomer 910, carbomer 934, carbomer 934P, carbomer 940, carbomer 941, carboxymethyl hydroxyethyl cellulose, carboxymethyl hydroxypropyl guar, carrageenan, cellulose, cellulose gum, cetearyl alcohol, cetyl alcohol, corn starch, damar, dextrin, dibenzlidine sorbitol, ethylene dihydrogenated tallowamide, ethylene diolamide, ethylene distearamide, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, isocetyl alcohol, isostearyl alcohol, karaya gum, kelp, lauryl alcohol, locust bean gum, magnesium aluminium silicate, magnesium silicate, magnesium trisilicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M, PEG-5M, polyacrylic acid, polyvinyl alcohol, potassium alginate, potassium aluminium polyacrylate, potassium carrageenan, potassium chloride, potassium sulfate, potato starch, propylene glycol alginate, sodium acrylate/vinyl alcohol copolymer, sodium carboxymethyl dextran, sodium carrageenan, sodium cellulose sulfate, sodium chloride, sodium polymethacylate, sodium silicoaluminate, sodium sulfate, stearalkonium bentotnite, stearalkonium hectorite, stearyl alcohol, tallow alcohol, TEA-hydrochloride, tragacanth gum, tridecyl alcohol, tromethamine magnesium aluminium silicate, wheat flour, wheat starch, xanthan gum, abietyl alcohol, acrylinoleic acid, aluminum behenate, aluminum caprylate, aluminum dilinoleate, aluminum salts, such as distearate, and aluminum isostearates, beeswax, behenamide, butadiene/acrylonitrile copolymer, C29-70 acid, calcium behenate, calcium stearate, candelilla wax, carnauba, ceresin, cholesterol, cholesterol hydroxystearate, coconut alcohol, copal, diglyceryl stearate malate, dihydroabietyl alcohol, dimethyl lauramine oleate, dodecanoic acid/cetearyl alcohol/glycol copolymer, erucamide, ethylcellulose, glyceryl triacetyl hydroxystearate, glyceryl tri-acetyl ricinolate, glycol dibehenate, glycol di-octanoate, glycol distearate, hexanediol distearate, hydrogenated C6-14 olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable oil, Japan wax, jojoba wax, lanolin alcohol, shea butter, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhyrdine copolymer, octyldodecyl stearoyl stearate, oleamide, oleostearine, ouricury wax, oxidized polyethylene, ozokerite, paraffin, pentaerythrityl hydrogenated rosinate, pentaerythrityl tetraoctanoate, pentaerythrityl rosinate, pentaerythrityl tetraabietate, pentaerythrityl tetrabehenate, pentaerythrityl tetraoleate, pentaerythrityl tetrastearate, ophthalmic anhydride/glycerin/glycidyl decanoate copolymer, ophthalmic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eiconsene copolymer, PVP/hexadecene copolymer, rice bran wax, stearlkonium bentonite, stearalkonium hectorite, stearamide, stearamide DEA-distearate, stearamide DIBA-stearate, stearamide MEA-stearate, stearone, stearyl erucamide, stearyl stearate, stearyl stearoyl stearate, synthetic beeswax, synthetic wax, trihydroxystearin, triisononanoin, triisostearin, tri-isostearyl trilinoleate, trilaurin, trilinoleic acid, trilinolein, trimyristin, triolein, tripalmitin, tristearin, zinc laurate, zinc myristate, zinc neodecanoate, zinc rosinate, and mixtures thereof. The gelling agents used in vehicles may be natural gelling agents such as natural gums, starches, pectins, agar and gelatin. Often, the gelling agents are based on polysaccharides or proteins Examples include but are not limited to guar gum, Xanthum gum, Alginic acid (E400), sodium alginate (E401), potassium alginate (E402), ammonium alginate (E403 alkanediol, may further comprise a humectant, such as, for example, glycerin, 1-2-propylene glycol, dipropylene glycol, polyethylene glycol, 1,3-butylene glycol, or 1,2,6-hexanetriol.

In certain non-limiting embodiments of the application, the antimicrobial effect of the inventive composition is achieved by a composition consisting of the combination of one or more botanical extract, solvent, and alkanediol, and optionally with a fruit acid or anti-inflammatory. In alternative embodiments of the application, one or more additional antimicrobial agent may be comprised, for example, where such antimicrobial agent may be selected from the group consisting of silver salts, iodophors, iodine, benzoic acid, dihydroacetic acid, propionic acid, sorbic acid, methyl paraben, ethyl paraben, propyl paraben, butyl paraben, cetrimide, benzalkonium chloride, dequalinium chloride, chlorhexidine, chloroeresol, chlorxylenol, benzyl alcohol, bronopol, chlorbutanol, phenoxyethanol, phenylethyl alcohol, 2,4-dichlorobenzyl alcohol, thiomersal, clindamycin, erythromycin, benzoyl peroxide, mupirocin, bacitracin, polymyxin B, neomycin, triclosan, parachlorometaxylene, foscarnet, miconazole, fluconazole, itriconazole, ketoconazole, silver sulfadiazine, octoxyglycerine, biguanides such as, but not limited to, chlorhexidine free base, chlorhexidine palmitate, chlorhexidine diphosphanilate, chlorhexidine digluconate, chlorhexidine diacetate, chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluorophosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine malate, chlorhexidine tartrate, chlorhexidine dimonoglycolate, chlorhexidine monodiglycolate, chlorhexidine dilactate, chlorhexidine di-α-hydroxyisobutyrate, chlorhexidine diglucoheptonate, chlorhexidine di-isothionate, chlorhexidine dibenzoate, chlorhexidine dicinnamate, chlorhexidine dimandelate, chlorhexidine di-isophthalate, chlorhexidine di-2-hydroxynapthoate, chlorhexidine embonate, and parahexamethylenebiguanide (“PHMB”).

In various non-limiting embodiments, a personal care product comprising a combination of one or more essential oil and/or IC together with one or more fruit acid may further comprise a neutralizing agent to neutralize carboxyl groups present in one or more other component, such as carboxyl groups in a thickening agent. Suitable neutralizing agents include diisopropylamine and triethanolamine.

In various non-limiting embodiments, the compositions used in a personal care product may further comprise a surfactant. The surfactant may be an anionic surfactant, a cationic surfactant, an ampholytic surfactant, or a nonionic surfactant. Examples of nonionic surfactants include polyethoxylates, fatty alcohols (e.g., ceteth-20 (a cetyl ether of polyethylene oxide having an average of about 20 ethylene oxide units) and other “BRIJ®” nonionic surfactants available from ICI Americas, Inc. (Wilmington, Del.)), cocamidopropyl betaine, alkyl phenols, fatty acid esters of sorbitol, sorbitan, or polyoxyethylene sorbitan. Suitable anionic surfactants include ammonium lauryl sulfate and lauryl ether sulfosuccinate.

In various non-limiting embodiments, a personal care product may comprise water.

In various non-limiting embodiments, the compositions used in a personal care product may further comprise a hydrogel comprising, for example, a compound such as hydroxypropylmethyl cellulose, cationic hydroxyethyl cellulose (U-care polymers), ethyl cellulose, hydroxypropyl cellulose, hydroxymethyl cellulose, carboxy methyl cellulose, polyethylene oxide (polyox resins), and chitosan pyrrolidone carboxylate (Kytomer PC).

In various non-limiting embodiments, a personal care product may further comprise an alcohol or a mixture of alcohols, for example, ethanol, isopropyl alcohol, n-propyl alcohol, and mixtures thereof; fatty alcohols, including, but not limited to, cetyl alcohol, myristol alcohol, stearyl alcohol, octyl alcohol, decyl alcohol and lauryl alcohol, and mixtures thereof; and hexanol.

In various non-limiting embodiments, the compositions used in a personal care product may further comprise a silicone polymer, for example one or more than one polydimethylsiloxane polymer (Dow Corning 225 Silicone Fluid), dimethiconol fluid in dimethicone (Dow Corning 1403 Silicone Fluid), cyclomethicone and dimethicone copolyl (Dow Corning 3225C Silicone Fluid), and silicone glycol (BASF 1066 DCG polyol).

In various non-limiting embodiments, the compositions used in a personal care product comprising a combination of one or more essential oil and/or IC together with one or more fruit acid may further comprise an emollient solvent such as a glycidyl ether having an alkyl chain up to and including 18 carbon molecules and ethoxylates and propoxylates thereof, a glyceryl ether having an alkyl chain up to and including 18 carbon molecules and ethoxylates and propoxylates thereof, a mono- or diglyceryl ether having an alkyl chain up to and including 18 carbon molecules and ethoxylates and propoxylates thereof, ethoxylate and propoxylate ethers, ethoxy diglycol esters, ethyl hexyl alcohol propoxylate, and propylene glycol esther ethoxylates and propoxylates, and Arlamol (Altas).

In various non-limiting embodiments, the compositions used in a personal care product comprising a combination of one or more essential oil and/or IC together with one or more fruit acid may further comprise additives such as dyes, fragrances, pH adjusters, including basic pH adjusters such as ammonia, mono-, di- and tri-alkyl amines, mono-, di- and tri-alkanolamines, alkali metal and alkaline earth metal hydroxides (e.g., ammonia, sodium hydroxide, potassium hydroxide, lithium hydroxide, monoethanolamine, triethylamine, isopropylamine, diethanolamine and triethanolamine); acid pH adjusters such as mineral acids and polycarboxylic acids (e.g., hydrochloric acid, nitric acid, phosphoric acid, sulfuric acid, citric acid, glycolic acid, and lactic acid); vitamins such as vitamin A, vitamin E and vitamin C; polyamine acids and salts, such as ethylenediamine tetraacidic acid (EDTA), preservatives such as German plus and DMDM hydantoin, and sunscreens such as aminobenzoic acid, arobenzone, cinoxate, diioxybenzone, homosalate, menthyl anthranilate, octocrylene, octyl methoxycinnamate, octyl salicylate, oxybenzoate, padimate O, phenylbenzimidazole, sulfonic acid, sulisobenzone, titanium dioxide, trolamine salicylate and zinc oxide.

In one set of non-limiting embodiments, the present subject matter provides for personal care compositions that are antimicrobial and anti-inflammatory (AM-AI) compositions for use in skin cleansers and topical creams.

The present subject matter also provides for rapidly acting AMI hand disinfectant lotions. The synergistic combination of GSE, Benzyl alcohol and 1,3 propanediol when used along with the anti inflammatory agent CRMN, edible plant extract (Kefiprotect®) and Pomegranate seed oil (PSO) exhibits additional synergistic activity.

The present subject matter also contemplates rapidly acting botanical AM-AI hand disinfectant lotions.

In specific, non-limiting embodiments, the present subject matter provides for the preparation of topical cream formulations containing anti-irritant, anti-inflammatory agents, gelling agents, and botanicals for minor cuts and wounds.

Antifungal activity of antifungal agents can be significantly enhanced by the use of synergistic combination of alcohols such as benzyl alcohol, fruit acids, and optionally biguanide and benzalkonium chloride.

The present subject matter also provides formulations containing GSE, benzyl alcohol, Zemea®, THC, and a coconut based phospholipid for alcohol-based hand sanitizer (AHS) compositions.

The present subject matter also provides for alcohol free botanical hand disinfectant lotions comprising between about 0.5% and 2% (w/w) benzyl alcohol; between about 0.3% and 2% (w/w) silicone fluid; between about 0.1% and 0.5% (w/w) zinc oxide; between about 0.1% and 2% (w/w) fruit acid, for example, lactic acid or citric acid; between about 0.05% and 1% (w/w) of one or more botanical selected from aloe gel, GSE, citrus extract, wasabi extract, willow bark extract, aspen bark extract and honey suckle extract; and between about 0.1% and 0.5% (w/w) of one or more essential oil selected from oregano oil, pomegranate oil, rosemary oil, lemongrass oil and cinnamon oil. Such compositions can optionally comprise between about 0.5% and 2% (w/w) farnesol, between about 0.05% and 0.3% (w/w) thymol, and/or between about 0.2% and 1% alkanediol.

Non-liming examples of alcohol free botanical hand disinfectant lotion formulations include the following:

Alcohol Free Botanical Hand Disinfectant Lotion A

| Ingredients | % w/w | Range (% w/w) |
|---|---|---|
| Water | 89.25 | 60-94.6 |
| Hydroxypropyl methyl- | 0.2 | 0.05-0.3 |
| cellulose stearoxy ether (Sangelose) | | |
| Allantoin | 0.3 | 0.1-0.3 |
| Polowax NF | 0.5 | 0.5-2.0 |
| Incroquat TMS | 0.5 | 0.5-4.0 |
| Stearyl alcohol | 0.5 | 0.5-3.0 |
| Vit. E acetate | 0.1 | 0.02-0.2 |
| Vit C | 0.1 | 0.02-0.2 |
| Zinc oxide | 0.3 | 0.1-0.5 |
| Glycerin | 2.0 | 1.0-3.0 |
| Butylene glycol | 1.0 | 0.5-3.0 |
| Benzyl alcohol | 1.0 | 0.5-2.0 |
| Silicone fluid | 1.0 | 0.3-2.0 |
| Lactic acid | 0.2 | 0.1-2.0 |
| Aloe gel | 1.0 | 0.01-2.0 |
| Symrelief | 0.05 | 0.01-1.0 |
| Pentanediol | 0 | 0-1.0 |
| Pomegranate oil | 0.3 | 0-1.0 |
| Oregano oil | 0.1 | 0.1-0.3 |
| Thymol | 0.1 | 0.05-0.3 |
| Rosemary oil | 0.1 | 0.1-1.0 |
| Lemongrass oil | 0.2 | 0.1-0.5 |
| Grapefruit seed/other Citrus extract | 0.5 | 0.3-1.0 |
| Wasabi extract | 0.3 | 0.1-0.5 |
| Benzoic acid | 0.2 | 0.2-0.5 |
| Sodium Benzoate | 0.2 | 0.2-0.5 |
| Adjust pH to 5.5-6.0 | | |

Alcohol Free Botanical Hand Disinfectant Lotion B

| Ingredients | % w/w | Range (% w/w) |
|---|---|---|
| Water | 86.90 | 60-94.6 |
| Hydroxypropyl methyl- | 0.2 | 0.05-0.3 |
| cellulose stearoxy ether (Sangelose) | | |
| Allantoin | 0.3 | 0.1-0.3 |
| Polowax NF | 0.5 | 0.5-2.0 |
| Incroquat TMS | 0.5 | 0.5-4.0 |
| Stearyl alcohol | 0.5 | 0.5-3.0 |
| Vit. E acetate | 0.1 | 0.02-0.2 |
| Vit C | 0.1 | 0.02-0.2 |
| Zinc oxide | 0.3 | 0.1-0.5 |
| Glycerin | 2.0 | 1.0-3.0 |
| Butylene glycol | 1.0 | 0.5-3.0 |
| Benzyl alcohol | 1.0 | 0.5-2.0 |
| Silicone fluid | 1.0 | 0.3-2.0 |
| Lactic acid | 0.2 | 0.1-2.0 |
| Aloe gel | 1.0 | 0.01-2.0 |
| Symrelief | 0.05 | 0.01-1.0 |
| Pentanediol | 1.0 | 0.2-1.0 |
| Pomegranate oil | 0.3 | 0-1.0 |
| Rosemary oil | 0.05 | 0.05-1.0 |
| Lemongrass oil | 0.1 | 0.05-0.5 |
| Cinnamon oil | 0.1 | 0.05-0.2 |
| Farnesol//Bisabolol | 0.5 | 0.3-2.0 |
| Thymol | 0.1 | 0.05-0.3 |
| Willow Bark extract | 1.0 | 0-2.0. |
| Aspen Bark extract | 0.5 | 0-2.0. |
| Wasabi extract | 0.3 | 0.1-0.5 |
| Benzoic acid | 0.2 | 0.2-0.5 |
| Sodium Benzoate | 0.2 | 0.2-0.5 |
| Adjust pH to 5.5-6.0 | | |

Alcohol Free Botanical Hand Disinfectant Lotion

| Ingredients | % w/w | Range (% w/w) |
|---|---|---|
| Water | 82.35 | 60-93.9 |
| Soft cat polymer | 0.3 | 0.1-0.5 |
| Allantoin | 0.3 | 0.1-0.3 |
| Polowax NF | 2.0 | 0.5-2.0 |
| Incroquat TMS | 2.0 | 0.5-4.0 |
| Stearyl alcohol | 2.0 | 0.5-3.0 |
| Isopropyl myristate | 1.0 | 0.5-2.0 |
| Arlacel 165 | 1.0 | 0.5-1.0 |
| Vit. E acetate | 0.1 | 0.02-0.5 |
| Zinc oxide | 0.3 | 0.1-0.5 |
| Glycerin | 2.0 | 1.0-3.0 |
| Butylene glycol | 1.0 | 0.5-3.0 |
| Lactic acid | 0.2 | 0.1-2.0 |
| Benzyl alcohol | 1.0 | 0.5-2.0 |
| Silicone fluid | 1.0 | 0.3-1.0 |
| Lactic acid | 0.2 | 0.1-2.0 |
| Aloe gel | 0.5 | 0.01-2.0 |
| Symrelief | 0.05 | 0.01-1.0 |
| Pentanediol | 1.0 | 0-1.0 |
| Pomegranate oil | 0.3 | 0-1.0 |
| Rosemary oil | 0.2 | 0.1-1.0 |
| Lemongrass oil | 0.2 | 0.1-0.5 |
| Honey suckle extract | 0.3 | 0.1-0.5 |
| Wasabi extract | 0.3 | 0.1-0.5 |
| Benzoic acid | 0.2 | 0.2-0.5 |
| Sodium Benzoate | 0.2 | 0.2-0.5 |
| Adjust pH to 5.5-6.0 | | |

The present subject matter also provides for aqueous botanical hand disinfectant lotions containing benzyl alcohol and botanicals. Non-liming examples of such formulations include the following:

Aqueous Botanical Hand Disinfectant Lotion A

| Ingredients | % w/w | Range (% w/w) |
|---|---|---|
| Water | 79.1 | 49.7-85.1 |
| Hydroxypropyl methyl- | 0.1 | 0.05-0.2 |
| cellulose stearoxy ether (Sangelose) | | |
| Allantoin | 0.3 | 0.1-0.3 |
| Polowax NF | 0.5 | 0.5-2.0 |
| Incroquat TMS | 0.5 | 0.5-4.0 |
| Stearyl alcohol | 0.5 | 0.5-3.0 |
| Vit. E acetate | 0.1 | 0.02-0.5 |
| Glycerin | 1.0 | 1.0-3.0 |
| Zinc oxide | 0.3 | 0.1-0.5 |
| Butylene glycol | 1.0 | 0.5-3.0 |
| Lactic acid | 0.2 | 0.1-2.0 |
| Benzyl alcohol | 1.0 | 0.5-2.0 |
| Silicone fluid | 1.0 | 0.3-2.0 |
| Aloe gel | 2.0 | 0.01-2.0 |
| Plant based alcohol (SDA40B from corn) | 10 | 10-20 |
| Pomegranate oil | 0.3 | 0-1.0 |
| Rosemary oil | 0.3 | 0.1-1.0 |
| Lemongrass oil | 0.2 | 0.1-0.5 |
| Thymol | 0.1 | 0.05-0.3 |
| Honey suckle extract | 0.3 | 0.1-1.0 |
| Grapefruit seed/other citrus extract | 0.5 | 0-0.5 |
| Wasabi extract | 0.3 | 0.0-0.5 |
| Benzoic acid | 0.2 | 0.2-0.5 |
| Sodium Benzoate | 0.2 | 0.2-0.5 |
| Adjust pH to 5.5-6.0 | | |

Aqueous Botanical Hand Disinfectant Lotion B

| Ingredients | % w/w | Range (% w/w) |
|---|---|---|
| Water | 73.5 | 49.5-84.97 |
| Hydroxypropyl methyl- | 0.1 | 0.05-0.2 |
| cellulose stearoxy ether (Sangelose) | | |
| Allantoin | 0.2 | 0.1-0.3 |
| Polowax NF | 0.5 | 0.5-2.0 |
| Incroquat TMS | 0.5 | 0.5-4.0 |
| Stearyl alcohol | 0.5 | 0.5-3.0 |
| Vit. E acetate | 0.1 | 0.02-0.5 |
| Zinc oxide | 0.3 | 0.1-0.3 |
| Glycerin | 1.0 | 1.0-5.0 |
| Benzyl alcohol | 1.0 | 0.5-2.0 |
| Silicone fluid | 2.0 | 0.3-1.0 |
| Aloe gel | 2.0 | 0.01-2.0 |
| Plant based alcohol (SDA40B from corn) | 15 | 10-20 |
| Pomegranate oil | 0.3 | 0-1.0 |
| Rosemary oil | 0.1 | 0-1.0 |
| Lemongrass oil | 0.1 | 0.1-0.5 |
| Thymol | 0.1 | 0.05-0.2 |
| Wasabi extract | 0.3 | 0.3-0.5 |
| Willow Bark extract | 0.5 | 0.-2.0 |
| Aspen Bark extract | 0.5 | 0.-2.0 |
| Farnesol/Bisabolol | 0.5 | 0.3-1.0 |
| Cationic surfactant | 0.5 | 0.3-1.0 |
| Benzoic acid | 0.2 | 0.2-0.5 |
| Sodium Benzoate | 0.2 | 0.2-0.5 |
| Adjust pH to 5.5-6.0 | | |

Aqueous Botanical Hand Disinfectant Lotion 1

| Ingredients | % w/w | Range (% w/w) |
|---|---|---|
| Water | 72.3 | 49.2-84.1 |
| Allantoin | 0.3 | 0.1-0.3 |
| Polowax NF | 2.0 | 0.5-2.0 |
| Incroquat TMS | 2.0 | 0.5-4.0 |
| Stearyl alcohol | 2.0 | 0.5-3.0 |
| Isopropyl myristate | 1.0 | 0.5-2.0 |
| Arlacel 165 | 1.0 | 0.5-1.0 |
| Vit. E acetate | 0.1 | 0.02-0.5 |
| Zinc oxide | 0.3 | 0.1-0.5 |
| Glycerin | 2.0 | 1.0-3.0 |
| Butylene glycol | 1.0 | 0.5-3.0 |
| Lactic acid | 0.2 | 0.1-2.0 |
| Benzyl alcohol | 1.0 | 0.5-2.0 |
| Silicone fluid | 1.0 | 0.3-1.0 |
| Aloe gel | 2.0 | 0.01-2.0 |
| Plant based alcohol | 10 | 10-20 |
| (SDA40B from corn) | | |
| Pomegranate oil | 0.3 | 0-1.0 |
| Rosemary oil | 0.3 | 0.3-1.0 |
| Lemongrass oil | 0.2 | 0.1-0.5 |
| Grapefruit seed extract | 0.3 | 0-0.5 |
| Wasabi extract | 0.3 | 0-0.5 |
| Benzoic acid | 0.2 | 0.2-0.5 |
| Sodium Benzoate | 0.2 | 0.2-0.5 |
| Adjust pH to 5.5-6.0 | | |

Aqueous Botanical Hand Disinfectant Lotion 2

| Ingredients | % w/w | Range (% w/w) |
|---|---|---|
| Water | 73.15 | 52.3-85.3 |
| Hydroxypropyl methyl- | 0.1 | 0.05-0.2 |
| cellulose stearoxy ether (Sangelose) | | |
| Allantoin | 0.2 | 0.1-0.3 |
| Polowax NF | 0.5 | 0.5-2.0 |
| Incroquat TMS | 0.5 | 0.5-4.0 |
| Stearyl alcohol | 0.5 | 0.5-3.0 |
| Vit. E acetate | 0.1 | 0.02-0.5 |
| Zinc oxide | 0.1 | 0.1-0.5 |
| Glycerin | 2.0 | 1.0-5.0 |
| Benzyl alcohol | 1.0 | 0.5-2.0 |
| Silicone fluid | 2.0 | 0.3-1.0 |
| Aloe gel | 2.0 | 0.01-2.0 |
| Plant based alcohol (SDA40B from corn) | 15 | 10-20 |
| Pomegranate oil | 0.3 | 0-1.0 |
| Rosemary oil | 0.3 | 0-1.0 |
| Lemongrass oil | 0.2 | 0.1-0.5 |
| Thymol | 0.1 | 0.05-0.2 |
| Galan gal oil | 0.05 | 0.02-0.5 |
| Grapefruit seed extract | 0.5 | 0-0.5 |
| Wasabi extract | 0 | 0-0.5 |
| Farnesol/Bisabolol | 0.5 | 0.3-1.0 |
| Cetrimonium chloride | 0.5 | 0.3-1.0 |
| Benzoic acid | 0.2 | 0.2-0.5 |
| Sodium Benzoate | 0.2 | 0.2-0.5 |
| Adjust pH to 5.5-6.0 | | |

The present subject matter also provides for aqueous botanical hand disinfectant foams containing benzyl alcohol and botanicals. Non-liming examples of such formulations include the following:

Aqueous Botanical Hand Disinfectant Foam A

| Ingredients | % w/w | Range (% w/w) |
|---|---|---|
| Water | 78.15 | 52.7-89.9 |
| Hydroxypropyl methyl- | 0.1 | 0.05-0.2 |
| cellulose stearoxy ether (Sangelose) | | |
| Allantoin | 0.3 | 0.2-0.5 |
| Grapefruit seed extract | 0.5 | 0.3-1.0 |
| Solubulizer (Peg-40 hydrogenated | 2.0 | 1-3 |
| Castor oil, Trideceth 9, water) | | |
| SDA-40 B (natural) | 10 | 5.0-20 |
| Benzyl alcohol | 1.0 | 0.1-2.0 |
| Pentylene glycol | 1.0 | 0.5-3.0 |
| Phenoxyethanol | 1.0 | 0.5-1.0 |
| Aloe leaf juice | 0.5 | 0.05-2.0 |
| Bisabolol | 0.05 | 0.025-0.1 |
| Wasabi extract | 0.3 | 0.2-0.5 |
| Honey suckle extract | 0.3 | 0.2-0.5 |
| Linalool | 0.1 | 0.05-0.3 |
| Lemon grass oil | 0.2 | 0.02-2.0 |
| Thymol | 0.1 | 0.05-0.2 |
| Phospholipid PTM (Croda) | 0.5 | 0-1.0 |
| Farnesol/Bisabolol | 0.5 | 0.3-1.0 |
| Cationic surfactant | 0.5 | 0.5-2.0 |
| Benzoic acid | 0.2 | 0.02-2.0 |
| Glucopon | 1.3 | 0.5--3.0 |
| Silicone (silsurf) | 1.4 | 0.5-2.0 |
| Adjust pH = 4.0-4.5 | | |

Aqueous Botanical hand Disinfectant Foam B

| Ingredients | % w/w | Range (% w/w) |
|---|---|---|
| Water | 80.65 | 54.7-90.8 |
| Hydroxypropyl methyl- | 0.1 | 0.05-0.2 |
| cellulose stearoxy ether (Sangelose) | | |
| Allantoin | 0.3 | 0.2-0.5 |
| Solubulizer (Peg-40 hydrogenated | 2.0 | 1-3 |
| Castor oil, Trideceth 9, water) | | |
| SDA-40 B (natural) | 10 | 5.0-20 |
| Benzyl alcohol | 1.0 | 0.1-2.0 |
| Pentylene glycol | 1.0 | 0.5-3.0 |
| Phenoxyethanol | 1.0 | 0.5-1.0 |
| Aloe leaf juice | 0.5 | 0.05-2.0 |
| Bisabolol | 0.05 | 0.025-0.1 |
| Linalool | 0.1 | 0.05-0.3 |
| Lemon grass oil | 0.1 | 0.02-2.0 |
| Farnesol | 0.5 | 0.3-2.0 |
| Thymol | 0.1 | 0.05-0.2 |
| Phospholipid PTM (Croda) | 0 | 0-1.0 |
| Benzoic acid | 0.2 | 0.02-2.0 |
| Cationic surfactant | 0.5 | 0.3-1.0 |
| Glucopon | 0.5 | 0.5-3.0 |
| Silicone (silsurf) | 1.4 | 0.5-2.0 |
| Adjust pH = 4.0-4.5 | | |

Aqueous Botanical Hand Disinfectant Foam 1

| Ingredients | % w/w | Range (% w/w) |
|---|---|---|
| Water | 81.65 | 58-91.5 |
| Kytamer (Chitoson complex) | 0.2 | 0.05-0.4 |
| Allantoin | 0.3 | 0.2-0.5 |
| Cetrimonium chloride | 0.5 | 0.1-1.0 |
| Honeysuckle extract | 0.5 | 0.3-1.0 |
| Solubulizer (Peg-40 hydrogented | 2.0 | 1-3 |
| Castor oil, Trideceth 9, water) | | |
| SDA-40 B (natural) | 10 | 5.0-20 |
| Benzyl alcohol | 1.0 | 0.1-2.0 |
| Phenoxyethanol | 1.0 | 0.5-1.0 |
| Aloe leaf juice | 0.5 | 0.05-2.0 |
| Bisabolol | 0.05 | 0.025-0.1 |
| Linalool | 0.1 | 0.05-0.3 |
| Lemon grass oil | 0.2 | 0.02-2.0 |
| Thymol | 0.1 | 0.05-0.2 |
| Galan gal oil | 0.05 | 0.02-0.5 |
| Phospholipid PTM (Croda) | 0 | 0-1.0 |
| Benzoic acid | 0.1 | 0.02-2.0 |
| Glucopon | 0.75 | 0.5-3.0 |
| Silicone (silsurf) | 1.0 | 0.5-2.0 |
| Adjust pH = 4.0-4.5 | | |
| or to pH = 5.5-6.0 | | |

Aqueous Botanical Hand Disinfectant Foam A1

| Ingredients | % w/w | Range (% w/w) |
|---|---|---|
| Water | 78.75 | 53.7-90.3 |
| Hydroxypropyl methyl- | 0.1 | 0.05-0.2 |
| cellulose stearoxy ether (Sangelose) | | |
| Allantoin | 0.3 | 0.2-0.5 |
| Grapefruit seed extract | 0.5 | 0.3-1.0 |
| Solubulizer (Peg-40 hydrogented | 2.0 | 1-3 |
| Castor oil, Trideceth 9, water) | | |
| SDA-40 B (natural) | 10 | 5.0-20 |
| Benzyl alcohol | 1.0 | 0.1-2.0 |
| Pentylene glycol | 1.0 | 0.5-3.0 |
| Phenoxyethanol | 1.0 | 0.5-1.0 |
| Aloe leaf juice | 0.5 | 0.05-2.0 |
| Bisabolol | 0.05 | 0.025-0.1 |
| Linalool | 0.1 | 0.05-0.3 |
| Lemon grass oil | 0.2 | 0.02-2.0 |
| Thymol | 0.1 | 0.05-0.2 |
| Phospholipid PTM (Croda) | 0.5 | 0-1.0 |
| Farnesol/Bisabolol | 0.5 | 0.3-1.0 |
| Dehyquart-CA surfactant | 0.5 | 0.5-2.0 |
| Benzoic acid | 0.2 | 0.02-2.0 |
| Glucopon | 1.3 | 0.5-3.0 |
| Silicone (silsurf) | 1.4 | 0.5-2.0 |
| Adjust pH = 4.0-4.5 | | |

Natural Alcohol Foam Hand Disinfectant

| Ingredients | % w/w | Range (% w/w) |
|---|---|---|
| Water | 23.2 | 15-30 |
| Hydroxypropyl methyl- | 0.05 | 0.05-0.2 |
| cellulose stearoxy ether (Sangelose) | | |
| Allantoin | 0.3 | 0.2-0.5 |
| Wasabi extract | 0.3 | 0.3-1.0 |
| Grapefruit seed extract | 0 | 0.0-1.0 |
| Honeysuckle extract | 0 | 0.0-1.0 |
| Corn derived SDA-40 B (natural) | 67 | 60.0-75.0 |
| Lemon grass oil | 0.2 | 0.05-1.0 |
| Veg. Glycerin | 2.0 | 0.5-3.0 |
| Benzyl alcohol | 1.0 | 0.5-3.0 |
| Benzoic acid | 0.2 | 0.05-1.0 |
| Bisabolol | 0.05 | 0.02-0.5 |
| Symsitive | 0.2 | 0.05-1.0 |
| Phenyl ethanol | 0.7 | 0.1-2.0 |
| Zemea (1,3-Propane diol) | 1.0 | 0.05-3.0 |
| Farnesol | 0.3 | 0.05-2.0 |
| Aloe leaf juice | 0.5 | 0.5-2.0 |
| Dow Corning 190 | 1.0 | 0.1-2.0 |
| Ultrapure MFB-10 | 2.0 | 0.5-3.0 |

Natural Alcohol Surgical Hand Disinfectant

| Ingredients | % w/w | Range (% w/w) |
|---|---|---|
| Water | 11.6 | 5.0-30.0 |
| Hydroxypropyl methyl- | 0.05 | 0.05-0.2 |
| cellulose stearoxy ether | | |
| (Sangelose) | | |
| Allantoin | 0.3 | 0.2-0.5 |
| Wasabi extract | 0.3 | 0.1-1.0 |
| Corn derived SDA-40 B (natural) | 79 | 70.0-85.0 |
| Lemon grass oil | 0.05 | 0.05-1.0 |
| Veg. Glycerin | 2.0 | 0.5-3.0 |
| Benzyl alcohol | 1.0 | 0.5-3.0 |
| Lactic acid | 0.2 | 0.05-1.0 |
| Farnesol | 1.0 | 0.05-2.0 |
| Sensiva (Ethylhexyl glycerin) | 2.0 | 0.05-3.0 |
| Bisabolol | 0.1 | 0.05-0.5 |
| Symsitive | 0.2 | 0.05-1.0 |
| Phenyl ethanol | 0.7 | 0.1-2.0 |
| Zemea (1,3-Propanediol) | 1.0 | 0.05-3.0 |
| Aloe leaf juice | 0.5 | 0.5-2.0 |

The present subject matter also provides for botanical disinfectant soaps containing benzyl alcohol and botanicals. Non-liming examples of such formulations include the following:

Botanical Disinfectant Soap A

| Ingredients | % w/w | Range (% w/w) |
|---|---|---|
| Water | 61.0 | 33.5-83 |
| Pluronic F-87 prill | 1.00 | 0.5-1.0 |
| Methocel E4 M | 0.2 | 0.1-0.5 |
| PolyoxWSR 205 | 0.3 | 0.1-0.5 |
| Glucopon | 2.0 | 2.0-3.0 |
| Dow corning 190 silicone | 1.0 | 0.5-3.0 |
| surfactant | | |
| Cocoamido propyl Betaine | 3.0 | 0-5.0 |
| SDA 40 B alcohol | 15.0 | 10-20 |
| Lemon grass oil | 0.2 | 0.05-2.0 |
| Orange oil | 0.1 | 0.01-0.5 |
| Benzyl alcohol | 2.0 | 0.5-3.0 |
| Honey suckle extract | 0.5 | 0.1-2.0 |
| Lactic acid | 0.2 | 0.1-2.0 |
| SDA 40 B | 12 | 2-20 |
| Veg. Glycerin | 1.0 | 0.5-2.0 |
| Aloe leaf juice | 0.5 | 0.5-2.0 |

Botanical Disinfectant Soap B

| Ingredients | % w/w | Range (% w/w) |
|---|---|---|
| Water | 60.5 | 32.5-82.7 |
| Pluronic F-87 prill | 1.00 | 0.5-1.0 |
| Methocel E4 M | 0.2 | 0.1-0.5 |
| PolyoxWSR 205 | 0.3 | 0.1-0.5 |
| Glucopon | 2.0 | 2.0-3.0 |
| Dow corning 190 silicone | 1.0 | 0.5-3.0 |
| surfactant | | |
| Cocoamido propyl Betaine | 3.0 | 0-5.0 |
| SDA 40 B alcohol | 15.0 | 10-20 |
| Lemon grass oil | 0.2 | 0.05-2.0 |
| Orange oil | 0.1 | 0.01-0.5 |
| Benzyl alcohol | 2.0 | 0.5-3.0 |
| Wasabi extract | 0.5 | 0.1-2.0 |
| Farnesol | 0.5 | 0.3-1.0 |
| Lactic acid | 0.2 | 0.1-2.0 |
| SDA 40 B | 12 | 2-20 |
| Veg. Glycerin | 1.0 | 0.5-2.0 |
| Aloe leaf juice | 0.5 | 0.5-2.0 |

Botanical Disinfectant Soap

| Ingredients | % w/w | Range (% w/w) |
|---|---|---|
| Water | 76.0 | 50-93.0 |
| Pluronic F-87 prill | 1.00 | 0.5-1.0 |
| Methocel E4 M | 0.2 | 0.1-0.5 |
| PolyoxWSR 205 | 0.3 | 0.1-0.5 |
| Glucopon | 2.0 | 2.0-3.0 |
| Dow corning 190 silicone | 1.0 | 0.5-3.0 |
| surfactant | | |
| Cocoamido propyl Betaine | 3.0 | 0-5.0 |
| Lemon grass oil | 0.2 | 0.05-2.0 |
| Orange oil | 0.1 | 0.01-0.5 |
| Benzyl alcohol | 2.0 | 0.5-3.0 |
| Honey suckle extract | 0.5 | 0.1-2.0 |
| Lactic acid | 0.2 | 0.1-2.0 |
| SDA 40 B | 12 | 2-20 |
| Veg. Glycerin | 1.0 | 0.5-2.0 |
| Aloe leaf juice | 0.5 | 0.5-2.0 |

### 4.14 Wound Healing

The compositions of the present subject matter may be used to treat wound healing or surface infections. In various non-limiting embodiments, the compositions of the present subject matter may be utilized in products such as topical creams and lotions, wound care products, burn wound cream, decubitous ulcer cream (with anti-inflammatory botanicals and the use of silver sulfadiazene as an anti-microbial agent), and therapeutic ointments. The compositions of the present subject matter may also be applied to wound care items, such as, but not limited to, wound healing ointments, wound coverings, burn wound cream, bandages, tape, and steri-strips, and medical articles such as medical gowns, caps, face masks, and shoe-covers, surgical drops, etc.

In various non-limiting embodiments, the products may further comprise a thickening and/or gelling agent such as stearyl alcohol, cationic hydroxy ethyl cellulose (Ucare; JR30), hydroxy propyl methyl cellulose, hydroxy propyl cellulose (Klucel), chitosan pyrrolidone carboxylate (Kytamer), behenyl alcohol, zinc stearate, emulsifying waxes, including but not limited to Incroquat and Polawax, an addition polymer of acrylic acid, a resin such as Carbopol® ETD™ 2020, guar gum, acacia, acrylates/steareth-20 methacrylate copolymer, agar, algin, alginic acid, ammonium acrylate co-polymers, ammonium alginate, ammonium chloride, ammonium sulfate, amylopectin, attapulgite, bentonite, C9-15 alcohols, calcium acetate, calcium alginate, calcium carrageenan, calcium chloride, caprylic alcohol, carbomer 910, carbomer 934, carbomer 934P, carbomer 940, carbomer 941, carboxymethyl hydroxyethyl cellulose, carboxymethyl hydroxypropyl guar, carrageenan, cellulose, cellulose gum, cetearyl alcohol, cetyl alcohol, corn starch, damar, dextrin, dibenzlidine sorbitol, ethylene dihydrogenated tallowamide, ethylene diolamide, ethylene distearamide, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, isocetyl alcohol, isostearyl alcohol, karaya gum, kelp, lauryl alcohol, locust bean gum, magnesium aluminium silicate, magnesium silicate, magnesium trisilicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M, PEG-5M, polyacrylic acid, polyvinyl alcohol, potassium alginate, potassium aluminium polyacrylate, potassium carrageenan, potassium chloride, potassium sulfate, potato starch, propylene glycol alginate, sodium acrylate/vinyl alcohol copolymer, sodium carboxymethyl dextran, sodium carrageenan, sodium cellulose sulfate, sodium chloride, sodium polymethacylate, sodium silicoaluminate, sodium sulfate, stearalkonium bentotnite, stearalkonium hectorite, stearyl alcohol, tallow alcohol, TEA-hydrochloride, tragacanth gum, tridecyl alcohol, tromethamine magnesium aluminium silicate, wheat flour, wheat starch, xanthan gum, abietyl alcohol, acrylinoleic acid, aluminum behenate, aluminum caprylate, aluminum dilinoleate, aluminum salts, such as distearate, and aluminum isostearates, beeswax, behenamide, butadiene/acrylonitrile copolymer, C29-70 acid, calcium behenate, calcium stearate, candelilla wax, carnauba, ceresin, cholesterol, cholesterol hydroxystearate, coconut alcohol, copal, diglyceryl stearate malate, dihydroabietyl alcohol, dimethyl lauramine oleate, dodecanoic acid/cetearyl alcohol/glycol copolymer, erucamide, ethylcellulose, glyceryl triacetyl hydroxystearate, glyceryl tri-acetyl ricinolate, glycol dibehenate, glycol di-octanoate, glycol distearate, hexanediol distearate, hydrogenated C6-14 olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable oil, Japan wax, jojoba wax, lanolin alcohol, shea butter, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhyrdine copolymer, octyldodecyl stearoyl stearate, oleamide, oleostearine, ouricury wax, oxidized polyethylene, ozokerite, paraffin, pentaerythrityl hydrogenated rosinate, pentaerythrityl tetraoctanoate, pentaerythrityl rosinate, pentaerythrityl tetraabietate, pentaerythrityl tetrabehenate, pentaerythrityl tetraoleate, pentaerythrityl tetrastearate, ophthalmic anhydride/glycerin/glycidyl decanoate copolymer, ophthalmic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eiconsene copolymer, PVP/hexadecene copolymer, rice bran wax, stearlkonium bentonite, stearalkonium hectorite, stearamide, stearamide DEA-distearate, stearamide DIBA-stearate, stearamide MEA-stearate, stearone, stearyl erucamide, stearyl stearate, stearyl stearoyl stearate, synthetic beeswax, synthetic wax, trihydroxystearin, triisononanoin, triisostearin, tri-isostearyl trilinoleate, trilaurin, trilinoleic acid, trilinolein, trimyristin, triolein, tripalmitin, tristearin, zinc laurate, zinc myristate, zinc neodecanoate, zinc rosinate, and mixtures thereof. The gelling agents used in vehicles may be natural gelling agents such as natural gums, starches, pectins, agar and gelatin. Often, the gelling agents are based on polysaccharides or proteins Examples include but are not limited to guar gum, Xanthum gum, Alginic acid (E400), sodium alginate (E401), potassium alginate (E402), ammonium alginate (E403), calcium alginate (E404, —polysaccharides from brown algae), Agar (E406, a polysaccharide obtained from red seaweeds), Carrageenan (E407, a polysaccharide obtained from red seaweeds), Locust bean gum (E410, a natural gum from the seeds of the Carob tree), Pectin (E440, a polysaccharide obtained from apple or citrus-fruit), and Gelatin (E441, made by partial hydrolysis of animal collagen).

Various embodiments may comprise a stabilizer. In a non-limiting example, sodium perborate is used as the stabilizing agent in an amount ranging from about 0.3 to about 1% w/w.

Various embodiments of the application may further comprise a surfactant. The surfactant may be an anionic surfactant, a cationic surfactant, an ampholytic surfactant, or a nonionic surfactant. Examples of nonionic surfactants include polyethoxylates, fatty alcohols (e.g., ceteth-20 (a cetyl ether of polyethylene oxide having an average of about 20 ethylene oxide units) and other “BRIJ®” nonionic surfactants available from ICI Americas, Inc. (Wilmington, Del.)), cocamidopropyl betaine, alkyl phenols, fatty acid esters of sorbitol, sorbitan, or polyoxyethylene sorbitan. Suitable anionic surfactants include ammonium lauryl sulfate and lauryl ether sulfosuccinate. Preferred surfactants include lauroyl ethylenediamine triacetic acid sodium salt, Pluronic F87, Masil SF-19 (BASF) and incromide.

Water used in the formulations described herein is preferably deionized water having a neutral pH.

Various embodiments of the application may comprise additional additives, including but not limited to a silicone fluid (such as dimethicone or cyclomethicone), a silicone emulsion, dyes, fragrances, pH adjusters, including basic pH adjusters such as ammonia, mono-, di- and tri-alkyl amines, mono-, di- and tri-alkanolamines, alkali metal and alkaline earth metal hydroxides (e.g., ammonia, sodium hydroxide, potassium hydroxide, lithium hydroxide, monoethanolamine, triethylamine, isopropylamine, diethanolamine and triethanolamine); acid pH adjusters such as mineral acids and polycarboxylic acids (e.g., hydrochloric acid, nitric acid, phosphoric acid, sulfuric acid, citric acid, glycolic acid, and lactic acid); vitamins such as vitamin A, vitamin E and vitamin C; polyamino acids and salts, such as ethylenediamine tetraacidic acid (EDTA), preservatives such as Germall plus and DMDM hydantoin, and sunscreens such as aminobenzoic acid, arobenzone, cinoxate, diioxybenzone, homosalate, menthyl anthranilate, octocrylene, octyl methoxycinnamate, octyl salicylate, oxybenzoate, padimate O, phenylbenzimidazole, sulfonic acid, sulisobenzone, titanium dioxide, and trolamine salicylate.

In specific non-limiting embodiments, the present subject matter provides for a wound healing topical cream containing silver sulfadiazine, an insoluble zinc salt, a soluble zinc salt and calendula oil. In another non-limiting embodiment, the present subject matter provides for a wound healing topical cream containing silver sulfadiazine, an insoluble zinc salt, a soluble zinc salt, calendula oil, and anti inflammatory agents such as a curcumin compound.

The present subject matter also provides for a topical antimicrobial, wound healing, anti-inflammatory cream containing silver sulfadiazine, an insoluble zinc salt, a soluble zinc salt, calendula oil, and synergistic combinations of curcumin compounds, benzyl alcohol, and 1,3 propanediol or octanediol or decanediol, which also enhance the antifungal activity.

In various embodiments, the compositions further contain a silver releasing agent. In other embodiments, the compositions further contain a stabilizer.

Non-limiting examples of cream products may further contain white petrolatum (2-20%), fatty alcohol (2-20%), emollient (1-10%), emulsifying agent (0.5-10%), humectant (2-15%), preservative (0.1-0.5%), and deionized or distilled water q.s. 100%. Fatty alcohols include stearyl, alcohol, cetyl alcohol, lauryl alcohol, myristyl alcohol, and other known fatty alcohols. Emollients include isopropyl myristate, lanolin, lanolin derivatives, isopropyl palmitate, isopropyl stearate and other known emollients. Emulsifying agents include sodium mono-oleate and polyoxyl 40 stearate. Humectants include propylene glycol, sorbitol, or glycerine or mixture thereof. Suitable water soluble preservatives include parabens, sorbic acid, benzoic acid, diazolidinyl urea, and iodopropylbutylcarbamate (Germal+).

The present subject matter also provides for antifungal diaper rash creams and ointments containing benzyl alcohol, botanicals and antifungal agents such as miconazole. Various topical antifungal agents may be used in the present subject matter, including but not limited to miconazole, oxiconazole, sulconazole, clotrimazole, econazole, ketoconozole, sertaconozol, fluconozole, and amphotericin B.

The present subject matter also provides for wound healing gel formulations containing benzyl alcohol and botanicals. Non-liming examples of such formulations include the following:

Botanical Wound Healing Gel

| Ingredients | % w/w | Range (% w/w) |
|---|---|---|
| Echinacea purpuria extract | 0.3 | 0.1-1.0 |
| Pomegranate oil | 0.3 | 0.1-1.0 |
| Rosemary oil | 0.3 | 0.1-1.0 |
| Lemongrass oil | 0.1 | 0.1-0.5 |
| Cinnamon oil | 0.2 | 0-0.5 |
| Thymol | 0.1 | 0.05-0.2 |
| Benzoic acid | 0.2 | 0.2-0.5 |
| Sodium Benzoate | 0.2 | 0.2-0.5 |
| Curcumin | 0.5 | 0.05-0.7 |
| Oatmeal | 1.0 | 0.5-5.0 |
| Ascorbic acid (Vitamin C) | 1.0 | 0.5-2.0 |
| Calendula Oil | 0.5 | 0.3-1.0 |
| Benzyl alcohol | 0.5 | 0.1-0.5 |
| Zinc Oxide | 0.3 | 0.2-1.0 |
| Zinc stearate | 0.3 | 0.2-1.0 |
| Glycerin | 10.0 | 5.0-20.0 |
| Buckthorn oil | 0.3 | 0-0.5 |
| Virgin coconut oil | 1.0 | 0-2.0 |
| Almond oil | 0.3 | 0.1-1.0 |
| Water | 80.3 | 54.1-91.9 |
| Aloe gel | 2.0 | 0.25-5.0 |
| Hydroxypropylmethylcellulose and derivatives | 0.3 | 0.1-1.0 |
| Adjust pH to 5.0-6.0 | | |

### 4.15 Veterinary Products

In a subset of non-limiting embodiments, the present subject matter provides for veterinary products for care of any domestic animal, including but not limited to cats, dogs, birds, rodents, rabbits, horses, cows and cattle, sheep, goats, etc.

Non-limiting examples of veterinary care products which may utilize the compositions of the present subject matter include pet shampoo, pet cleansing wipes including body wipes, ear wipes, and eye wipes, dental wipes, toothpaste, ear cleaning liquid, cage cleaner, surface cleaner for housebreaking accidents, topical creams, ointments, teat dip therapeutic for mastitis and liquid to be applied to pet's skin (as in a “body splash”).

Veterinary care compositions according to the application may further comprise one or (preferably) more than one component selected from the group consisting of emollients, stabilizing agents, thickening agents, humectants, antimicrobial agents, neutralizing agents, surfactants, water, silicone polymers, alcohols, and hydrogels, anti-inflammatory agents, wound healing agents, salicylic acid, as well as additional components as may be known in the art.

Specific, non-limiting examples of additional components which may be comprised in pet care products include the components listed above for personal care products.

In certain non-limiting embodiments of the application, the compositions may be prepared for teat dip to treat mastitis. The anti-irritants used for teat dip may include but are not limited to zinc salts with panthenol, or Bisabolol with ginger root extract (symrelief), or symrelief with a zinc salt. The gelling agents in the vehicle may include but are not limited to natural gelling agents such as natural gums, starches, pectins, agar and gelatin. Antimicrobial botanicals may include but are not limited to lemongrass oil, orange oil and fruit acids such as citric and lactic acid, phenoxyethanol (constituent of sage oil).

### 4.16 Household/Industrial Products

In a subset of non-limiting embodiments, the present subject matter provides for household/industrial products comprising the formulations outlined above.

Non-limiting embodiments of household/industrial products which may utilize the compositions of the present subject matter include householder cleaners such as concentrated liquid cleaners and spray cleaners, cleaning wipes, dish washing liquid, dish washer detergent, spray-mop liquid, furniture polish, indoor paint, outdoor paint, dusting spray, laundry detergent, fabric softener, rug/fabric cleaner, window and glass cleaner, toilet bowl cleaner, liquid/cream cleanser, etc. In a particular embodiment, the compositions of the present subject matter may be used in a food wash product, designed to clean fruits and vegetables prior to consumption. “Household products” are products, other than personal care products, that would be used by individual consumers. “Industrial products” refers to products that are used in industry.

Household-industrial compositions according to the application may further comprise one or (preferably) more than one component selected from the group consisting of surfactants, builders (e.g., sequestering builders, precipitating builders, ion exchange builders), solvents, thickeners, abrasives, acids, bases (alkalis), antimicrobial agents, soaps, bleaching agents, enzymes, preservatives, and sudsing agents, as well as additional components as may be known in the art.

In various non-limiting embodiments, the compositions may further comprise a surfactant, for example, but not limited to, an anionic surfactant such as an alkyl sulfate, an alkyldiphenyloxide disulfonate salt (e.g., the DOWFAX series by the Dow Chemical Company), an alkylbenzenesulfonate, an alcohol ethoxysulfate; a cationic surfactant; a non-ionic surfactant, such as a secondary alcohol ethoxylate (e.g., the TERGITAOL series by the Dow Chemical Company) or an alkyl polyglucoside (e.g., the TRITON series by the Dow Chemical Company); or an amphoteric surfactant such as an imidazoline or betaine compound.

In various non-limiting embodiments, the compositions may further comprise a solvent, for example, but not limited to, water, an alcohol such as methanol, ethanol, isopropyl alcohol, or butanol; a hydrocarbon such as an aromatic hydrocarbon, propylene glycol, methylene chloride, acetone, a petroleum distillate, and/or a glycol ether.

In various non-limiting embodiments, the compositions used in a household/industrial product may further comprise a thickener, for example, but not limited to, a polyethylene glycol, a methoxypolyethylene glycol, and/or hydroxyethyl cellulose.

In various non-limiting embodiments, the compositions used in a household/industrial product may further comprise an abrasive, such as, but not limited to, silica, feldspar or calcite.

In various non-limiting embodiments, the compositions used in a household/industrial product may further comprise an acid, such as, but not limited to, acetic acid, hydroacetic acid, phosphoric acid or hydrochloric acid.

In various non-limiting embodiments, the compositions used in a household/industrial product may further comprise a base (alkali) such as, but not limited to, ammonia or sodium bicarbonate.

In various non-limiting embodiments, the compositions used in a household/industrial product may further comprise an antimicrobial agent, for example, but not limited to, compounds as set forth above for personal care compositions, and also pine oil and sodium hypochlorite.

In various non-limiting embodiments, the compositions used in a household/industrial product may further comprise a bleaching agent, for example, but not limited to, sodium hypochlorite, hydrogen peroxide, sodium percarbonate and sodium perborate.

In various non-limiting embodiments, the compositions used in a household/industrial product may further comprise an enzyme, such as, but not limited to, a protease or a lipase.

In various non-limiting embodiments, the compositions used in a household/industrial product may further comprise a preservative, such as, but not limited to, butylated hydroxytoluene, glutaraldehyde, and EDTA.

In various non-limiting embodiments, the compositions used in a household/industrial product may further comprise a sudsing agent, such as, but not limited to, diethanolamine or triethanolamine.

In specific, non-limiting embodiments, the present subject matter provides for the following surface cleaners, having concentrations of active ingredients as well as concentrated stock solutions of these formulations which may be diluted to achieve the respective concentrations.

In a subset of non-limiting embodiments, the present subject matter provides for preservatives and surface disinfectants comprising the formulations outlined above. In non-limiting embodiments, preservatives and surface disinfectants of the present subject matter include the following.

Botanical Preservative A

| Ingredient | % w/w | Range (% w/w) |
|---|---|---|
| Benzyl alcohol | 73.6 | 45.0-82.0 |
| Thymol | 7.4 | 5.0-10.0 |
| Galanga oil | 7.4 | 5.0-10.0 |
| Cinnamon oil | 2.9 | 2.0-10.0 |
| Lemon grass oil | 1.4 | 1.0-5.0 |
| Wasabi extract | 7.3 | 5.0-20 |

Use level is 0.20 to 1.0%

Botanical Surface Disinfectant A

| Ingredients | % w/w | Range (% w/w) |
|---|---|---|
| Lemongrass | 0.5 | 0.5-2.5 |
| Pine oil | 0.1 | 0.1-1.0 |
| Glucopon 215 UP | 5.0 | 5-25 |
| Citric acid | 2.0 | 2-10 |
| SDA 3C | 7.0 | 2.5-35 |
| Benzyl alcohol | 5.4 | 2.5-27 |
| Water | 80 | 0-90 |

Botanical Surface Disinfectant B

| Ingredients | % w/w | Range (% w/w) |
|---|---|---|
| Lemongrass | 0.5 | 0.5-2.5 |
| Pine oil | 0.1 | 0.1-1.0 |
| Glucopon 215 UP | 5.0 | 5-25 |
| Citric acid | 2.0 | 2-10 |
| Benzyl alcohol | 17 | 10-30 |
| Water | 75.4 | 31.5-69.4 |

Hard Surface Disinfectant C

| Ingredients | % w/w | Range (% w/w) |
|---|---|---|
| Sodium hypochlorite | 1.0 | 0.5-3.0 |
| Lemongrass | 0.5 | 0.5-2.5 |
| Pine oil | 0.1 | 0.1-1.0 |
| Glucopon 215 UP | 5.0 | 5-25 |
| Citric acid | 2.0 | 2-10 |
| Water | 81.4 | 28.5-86.9 |
| Benzyl alcohol | 10 | 5-30 |

Botanical Surface Disinfectant LG-50

| Ingredients | % w/w | Range (% w/w) |
|---|---|---|
| Lemongrass | 2.5 | 2.5-10.0 |
| Pine oil | 0.5 | 0.5-5.0 |
| Glucopon 215 UP | 25 | 25-50 |
| Citric acid | 10 | 10-20 |
| SDA 3C | 35 | 20-40 |
| Water | 27 | 20-40 |

### 4.17 Medical Devices

In a subset of non-limiting embodiments, the present subject matter provides for medical devices comprising the formulations outlined above.

Implantation of a medical device produces rapid inflammatory reaction at the implantation site. This may result in the formation of a biofilm on the surface of the medical device. The biofilm on the surface of a medical device serves as a receptor for microbes resulting in microbial adhesion. Prevention of inflammation around the implanted medical device can prevent bacterial adherence on the device. This may be achieved by maintaining an inflammation and infection-free environment around the device by coating and/or impregnating the device with anti inflammatory agents and antimicrobials.

Anti-inflammatory antimicrobial compositions comprising synergistic combination of benzyl alcohol, 1,3 propanediol and THC (with or without other antimicrobials such as chlorhexidine and silver salts) can be used to coat or impregnate medical devices such as catheters, wound dressing, soft tissue patches, etc.

### 4.18 Pesticides

In a subset of non-limiting embodiments, the present subject matter provides for pesticides comprising the formulations outlined above. In non-limiting embodiments, pesticides of the present subject matter include the following.

Botanical Plant Pesticide

| Ingredients | % w/w | Range (% w/w) |
|---|---|---|
| Benzyl alcohol | 10 | 5-20 |
| Tobacco extract | 0.2 | 0.01-2.0 |
| Lemongrass oil | 1.0 | 0.01-2.0 |
| Curry leaf oil | 0.5 | 0.05-2.0 |
| Cedar wood oil | 2.0 | 0.5-4.0 |
| Cocoamido propyl betaine | 2.0 | 0.5-5.0 |
| Water | 82.8 | 60-93.83 |
| Tea extract | 0.5 | 0.1-3.0 |
| Solubilizer | 1.0 | 0.0-2.0 |

For use, the above composition can be diluted 1 to 10 with, for example water.

### 5. EXAMPLES

The detailed description hereby incorporates, by reference, the specific working examples of the application set forth below.

The working examples sometimes refer to Dial® soap. Dial® soap is a commercially sold liquid soap, where Dial® Antibacterial hand soap comprises, as active agent, 0.15 percent triclosan, and the inactive agents are water, sodium laureth sulfate, ammonium lauryl sulfate, decyl glucoside, cocamidopropyl betaine, glycerine, sodium chloride, PEG-18 gylceryl oleate/cocoate, fragrance, cocamide MEA, DMDM hydantoin, tetrasodium ethylene diamine tetracetic acid and colors.

### Example 1

### Antimicrobial Efficacy of an Aqueous Botanical Hand Disinfectant Foam A: Rapid (15 Second) and Broad Spectrum Antibacterial Efficacy

The FDA TFM method (modified slightly by using lower volumes of sanitizer and microbe culture, but in the same proportion) for testing the efficacy to rapidly kill bacteria was used. The rapid antibacterial activity (15 seconds exposure) of the hand foam of the present subject matter was tested against various organisms listed in Table 1.

### Method:

Briefly, hand foam (0.8 ml) was placed in a sterile culture tube and 0.2 ml of 10⁹ cfu/ml of test bacterial culture (or 10⁷ cfu/ml of Candida culture), diluted 1:1 with bovine adult serum, was added and mixed. After 15 s, the antimicrobial activity was stopped with drug neutralizing fluid (DNF). Serial dilutions were made with DNF, and aliquots were subcultured on trypticase soy agar to determine the number of microbial colonies per sample. For controls, phosphate buffered saline (PBS) containing the same bacterial cultures were used instead of the test formulation. The antibacterial activity was calculated by determining the difference in log₁₀ colony-forming units (cfu) between control (Phosphate buffer saline) and test samples (i.e., log₁₀ reduction). 3 experiments, each containing triplicate samples in each group, were performed. For comparison, triclosan and PCMX containing soap, and an alcohol (62%) hand sanitizer, were tested similarly at the same time.

TABLE 1
Rapid and broad antimicrobial efficacy of an aqueous botanical hand
disinfectant foam and Purell by the FDA TFM method*

| | % kill | |
|---|---|---|
| | Purell | Aqueous Botanical hand |
| Organism | (62% Ethyl Alcohol) | disinfectant foam |
| S. aureus | >99.99 | >99.99 |
| ATCC# 6538 | | |
| P. aeruginosa | >99.99 | >99.9 |
| ATCC#15442 | | |
| E. coli | >99.999 | >99.999 |
| ATCC#35218 | | |
| MRSA | >99.99 | >99.9 |
| ATCC#4716 | | |
| S. epidermidis | ND | 99.9 |
| ATCC# 35983 | | |
| E. faecalis | ND | >99.9 |
| ATCC# 29212 | | |
| VREF | ND | 99.9 |
| Clinical isolate | | |
| S. marcescens | ND | >99.999 |
| ATCC# 14756 | | |
| A. baumanni | ND | 99.9 |
| Clinical isolate | | |
| E. aerogenes | ND | >99.999 |
| ATCC#14053 | | |
| K. pneumonia | >99.99 | >99.999 |
| ATCC# 13883 | | |
| S. typhimurium | ND | >99.999 |
| ATCC#14028 | | |
| C. albicans | ND | >99.999 |
| ATCC#10231 | | |

ND = Not done
*FDA TFM = Tentative Final Monograph; ATCC = American Type Culture Collection; MRSA = Methicillin-resistant S. aureus; VREF = Vancomycin-resistant E. faecalis. The control growth ranged from 5 × 10⁸ to 1 × 10⁹ cfu/ml. Results are the average of twelve samples in each group.

### Example 2

### Rapid and Persistent Antibacterial Efficacy of an Aqueous Botanical Hand Disinfectant foamA

A pigskin test method was used to examine the antimicrobial efficacy of an aqueous botanical hand disinfectant. The pigskin test simulates the Volunteer method of the American Society for Testing and Materials (ASTM) E-1174 (Test organism: S. aureus)

### Method:

3 cm² pieces of pigskins were prepared and each piece was mounted on a petri dish. For the baseline count determinations, 2 skin samples were rinsed under running tap water for 30 seconds and then lathered using 0.5 ml of non-antibacterial soap by rubbing both pieces together for 30 seconds. The pair was then rinsed under running tap water for 30 seconds and blot dried. Each piece of pigskin was contaminated with 15 μl of 10⁷ cfu/ml S. aureus culture and rubbed against each other for 30 seconds and dried for 30 seconds. The organisms were recovered from the skin by rinsing each piece with 10 ml of DNF for 15 seconds and collecting the wash. Aliquots from serial dilutions were uniformly spread on TSA plates, incubated for 24-48 hours at 37° C. To evaluate the efficacy of the test sanitizers, the same skin pieces used for baseline count determinations were rinsed under running tap water, washed with non-antibacterial soap, and recontaminated. After the 30-second drying period, 0.5 ml of the test sanitizer was applied on the skin and lathered for 30 seconds. After rinsing, the bacteria were recovered as described above. The contamination and sanitizer applications were repeated an additional 9 times. The bacterial counts after 1 and 10 applications were determined and the difference in log₁₀ colony-forming units (cfu) between the baseline and test samples were calculated (log₁₀ reduction).

TABLE 2
Rapid and persistent antibacterial efficacy of hand disinfectant foam A
after repeated applications by the pig skin method (test organisms
S. aureus (ATCC 6538))

| | Log₁₀ reduction from |
|---|---|
| | control growth, mean ± SD |
| Test Soap | S. aureus |
| Alcohol hand sanitizer (Purell) | |
| After 1^{st} application | 1.8 ± 0.08 |
| After 10^{th} application | 1.9 ± 0.05 |
| Aqueous botanical hand disinfectant foam A | |
| After 1^{st} application | 2.6 ± 0.06 |
| After 10^{th} application | 3.0 ± 0.05 |

Control counts ranged from 1 × 10⁵ to 5 × 10⁵ cfu/ml. Results are the average of twelve samples in each group.

Conclusion: As per US FDA-TFM criteria, the required log₁₀ reduction from control counts is 2.0 log after a first application for rapid antimicrobial activity, and 3.0 log after tenth application for persistent antimicrobial activity. Unlike the alcohol sanitizer Purell, an aqueous botanical hand disinfectant foam of the present subject matter exhibits the required US FDA-TFM criteria log reduction.

### Example 3

### Evaluation of the Antimicrobial Efficacy of a Surface Disinfectant: In Vitro Rapid Kill (15 Seconds) of S. aureus

### Method:

Surfactant (0.8 ml) was placed in a sterile culture tube and 0.2 ml of 10⁹ cfu/ml of test bacterial culture diluted 1:1 with bovine adult serum was added and mixed. After 15 s, the antimicrobial activity was stopped with drug neutralizing fluid (DNF). Serial dilutions were made with DNF and aliquots were subcultured on trypticase soy agar to determine the number of microbial colonies per sample. For controls, phosphate buffered saline (PBS) containing the same bacterial cultures was used instead of the test formulation. The antibacterial activity was calculated by determining the difference in log₁₀ colony-forming units (cfu) between control (phosphate buffer saline) and test samples (i.e., log₁₀ reduction).

TABLE 3

| | Log reduction from | |
|---|---|---|
| Compositions | control growth | % kill |
| Surface disinfectant LG-50 | 7.0 | 100 |
| Proquart (Clorox)* | 3.85 | 99.95 |
| (1 oz diluted to 1 Gallon) | | |
| Proquart (Clorox)* | 4.26 | 99.99 |
| (2 oz diluted to 1 Gallon) | | |

*These contain long chain quaternary ammonium compounds
Control bacterial counts: 8.5-9.9 log₁₀
Dilutions of Proquart were made as described in the instructions on the product label: Proquart 0.17% quaternary ammonium compound (1 oz. and 2 oz.) was diluted to 1 gallon.

Conclusion. The botanical surface disinfectant showed higher antimicrobial efficacy than Proquart.

### Example 4

### Efficacy of Disinfectants on Bacteria Dried on Ceramic Tiles Following a 10 Minute Exposure to Compositions of the Application

### Method:

In this experiment, S. aureus, MRSA and E. coli were used as the test organisms. Ceramic tiles with dimensions of 5×15 cm² were cleaned first with water and then with 70% ethanol and dried. Approximately 0.1 ml of 1×10⁷ cfulml of each microorganism was spread evenly on the surfaces of the tiles using a sterile glass rod, and allowed to dry for 1 hour. 0.3 ml of surface cleanser of the present subject matter was spread onto the tiles and allowed to dry at room temperature for 10 minutes. The tile surface was rinsed using 9.7 ml of Drug neutralizing fluid (DNF) into a sterile Petri dish to recover the remaining microorganisms on the tile. The recovered media was serially diluted, plated on TSA plates, and incubated at 37° C. for 18-24 hrs after which time colony counts were determined. For control, the same procedure was followed, except 0.3 ml of phosphate buffered saline was used instead of surface cleanser. Proquart, a commercial disinfectant containing quaternary ammonium compounds (10.85% quaternary ammonium compounds), from Clorox company was also evaluated simultaneously for comparison.

TABLE 4
Efficacy of a 10 minute application of LG-50 surfactants on bacteria
dried on ceramic tiles

| | % kill | | |
|---|---|---|---|
| Composition | S. aureus | MRSA | E. coli |
| Surface disinfectant LG-50 | 99.999 | 99.99 | 99.9 |
| (1 oz diluted to 5 oz) | | | |
| Proquart | 99.0 | 99.0 | 99.0 |
| (1 oz diluted to 1 Gallon) | | | |
| Proquart | 99.9 | 99.9 | 99.0 |
| (2 oz diluted to 1 Gallon) | | | |

Control growth ranged from 5 × 10⁵-1 × 10⁶.
Dilutions of Proquart were made as described in the instructions on the product label: Proquart 0.17% quaternary ammonium compound (1 oz. and 2 oz.) was diluted to 1 gallon.
LG 50 comprising 0.12% essential oils (1 oz) was diluted to 5 oz.

Conclusion. The botanical surface disinfectant showed higher antimicrobial efficacy than Proquart.

Various patent and non-patent publications are cited herein, the contents of which are hereby incorporated by reference in their entireties.

We claim:

1. A rapidly acting antimicrobial composition comprising from about 0.5 to about 50% (w/w) benzyl alcohol, from about 0.5 to about 50% (w/w) 1,3 propanediol, from about 0.5 to about 10% (w/w) botanical, from about 0 to about 20% (w/w) fruit acid, from about 2.5 to about 10% (w/w) alkanediol, and from about 5 to about 80% (w/w) solvent.

2. The rapidly acting antimicrobial composition of claim 1, wherein the botanical comprises essential oil, plant extract, fruit extract, individual constituents thereof, or combinations thereof.

3. A personal care product comprising the composition of claim 1.

4. The personal care product of claim 3, wherein the composition is used at a concentration ranging from about 1 to about 10% (w/w) in the personal care product.

5. A method of treating acne using a topical ointment comprising a preservative composition comprising synergistically effective amounts of:
(a) botanical;
(b) benzyl alcohol; and
(c) 1,3 propanediol,
wherein said amount of botanical to benzyl alcohol is present as a ratio of 1:1 to 1:12, and wherein the composition pH ranges from about 3-5, and wherein the composition further comprises salicylic acid.

6. An antifungal topical cream comprising a synergistic combination of from about 0.5 to about 5% (w/w) benzyl alcohol and one or more compounds selected from the group consisting of
(a) from about 0.3 to about 5.0% (w/w) 1,3 propanediol and its derivatives;
(b) from about 0.04 to about 0.5% (w/w) of one or more botanical selected from the group consisting of curcumin compounds;
(c) from about 0.04 to about 1.05% (w/w) essential oil, fruit extract, plant extract or combinations thereof;
(d) from about 0.2 to about 2.0% (w/w) fruit acid; and
(e) from about 0.5 to about 2.0% (w/w) alkanediol.

7. An antimicrobial composition comprising:
(a) from about 0.5 to about 10% (w/w) benzyl alcohol or its derivatives; and
(b) from about 0.2 to about 4.0% (w/w) botanical extract,
wherein the combination of benzyl alcohol and botanical extract exhibits synergistic activity.

8. The antimicrobial composition of claim 7, further comprising fruit acid and essential oil.

9. The antimicrobial composition of claim 8, wherein the fruit acid includes organic acid.

10. The antimicrobial composition of claim 9, wherein the organic acid comprises benzoic acid and its derivatives.

11. The antimicrobial composition of claim 9, wherein the organic acid is present in amounts ranging from about 0.1 to about 2.0% (w/w).

12. The antimicrobial composition of claim 8, wherein the fruit acid is selected from the group consisting of lactic acid, citric acid, and combinations thereof.

13. The antimicrobial composition of claim 8, wherein the essential oil is selected from the group consisting of lemongrass, cinnamon, basil, citronella, thyme, eucalyptus, oregano, peppermint, clove, menthol, thymol, eucalyptol and combinations thereof.

14. The antimicrobial composition of claim 7, further comprising synthetic antimicrobial.

15. The antimicrobial composition of claim 7, further comprising solvent.

16. The antimicrobial composition of claim 15, wherein the solvent comprises from about 5 to about 90% (w/w) aliphatic alcohol.

17. The antimicrobial composition of claim 15, wherein the solvent comprises from about 0.5 to about 5% (w/w) aromatic alcohol.

18. The antimicrobial composition of claim 15, wherein the solvent comprises from about 2 to about 10% (w/w) glycerin.

19. A food disinfectant cleanser comprising from about 0.5 to about 5.0% (w/w) benzyl alcohol, from about 0.2 to about 2.0% (w/w) fruit acid, from about 0.2 to about 1.0% (w/w) botanical extract, from about 0.1 to about 5.0% (w/w) surfactant, and water.
